(19) United States
(12) Patent Application Publication
(10) Pub. No.: US 2021/0375472 A1
(43) Pub. Date: Dec. 2, 2021

(54) METHODS AND SYSTEMS FOR DECISION SUPPORT

(71) Applicant: University of Washington, Washington, DC (US)

(72) Inventors: Joseph M. Czerniecki, Washington, DC (US); Daniel Norvell, Washington, DC (US)

(21) Appl. No.: 17/335,893

(22) Filed: Jun. 1, 2021

Related U.S. Application Data
(60) Provisional application No. 63/033,036, filed on Jun. 1, 2020.

(51) Int. Cl. G16H 50/30; G16H 10/60

(52) U.S. Cl. CPC G16H 50/30; G16H 10/60

(57) ABSTRACT

A system and methods for decision support are provided. In some aspects, the methods and systems determine a decision support model, determine one or more risk scores, and output the one or more risk scores.

### CROSS REFERENCE TO RELATED APPLICATION

This application claims priority to and the benefit of U.S. Provisional Application No. 63/033,036 filed Jun. 1, 2020, the entirety of which is incorporated herein by reference.

### GOVERNMENT SUPPORT LICENSE RIGHTS

This invention was made with government support under grant number 1 I01 RX002960-01 awarded by the Department of Veterans Affairs Office of Research and Development Merit Review. The government has certain rights in the invention.

### BACKGROUND

Amputation-level decision-making in the context of diabetes and peripheral artery disease (PAD) is extremely challenging. The preservation of mobility and its impact on quality of life are key considerations. The goal of preserving mobility has led to a focus on attempting the most distal amputation procedure possible: preserving the knee joint by performing a transtibial (TT) rather than a transfemoral (TF) amputation, or by attempting a limb salvage transmetatarsal (TM) amputation rather than a major amputation. Failure of primary healing results in the need for additional reamputation surgery and/or prolonged wound care with restricted ambulation which may adversely affect future mobility and increase the risk of death.

Patients undergoing amputation of the lower extremity due to the complications of peripheral artery disease and/or diabetes are at risk of treatment failure and the need for reamputation at a higher level. Amputation level (and specifically, an increase thereof), may refer to a series of amputations which progress from a distal end of a limb to a proximal end of the limb (e.g., towards the body) Further, patients who undergo lower extremity amputation secondary to complications of diabetes or peripheral artery disease have poor long-term survival and an increased risk of loss of mobility. Current approaches fail to provide patients and surgeons with individual (e.g., patient level), rather than population level estimates of reamputation risk, survival risk, or loss of mobility risk. Previous models associated with amputation stemming from the complications of diabetes or peripheral artery disease (PAD) have all been developed to predict operative risk (e.g., chances of mortality during the amputation operation itself).

There is a lack of evidence to assist surgeons and their patients in determining the magnitude of the probable risk of healing failure and need for additional surgery at each amputation level. Population risks, although important in understanding the outcomes of populations, are of limited value in shared decision-making (“SDM”) because they do not provide information about patient specific risk. Currently, amputation level decisions are made on the basis of a surgeon's clinical experience underpinned by immediate knowledge, but without objective information about patient specific long term outcomes. Further, simply accessing the medical literature is not a reliable method for improving compliance or changing clinician behavior. Finding documents can take a significant amount of time, and once identified, reading, interpreting, and acting on the guidelines can be much more complicated than using a tool (e.g., a decision support tool, or “DST”) that provides point of care patient specific guidance. Current clinical algorithms used to predict healing failure risk and future mobility have limitations, and there is a perceived need for a more individualized approach to decision-making and improved predictive data.

Operative and longer-term survival for patients undergoing lower extremity amputation because of critical limb ischaemia are poor, and mortality risks exceed those of the majority of cancer diagnosis. Communicating future mortality risk for individual patients is therefore a critical component of the amputation decision-making process. Current methods do not provide an estimate of risk of reamputation, loss of mobility, or long term survival that will better assist patients and physicians in shared decision making.

### SUMMARY

It is to be understood that both the following general description and the following detailed description are exemplary and explanatory only and are not restrictive. In an embodiment, provided are methods and systems for facilitating shared decision making via a decision support tool with regards to medical procedures such as amputation. Providing patients and physicians with patient specific risks regarding their individual outcome probabilities permits a more informed decision making process that can help ensure treatment methodologies and outcomes suit the patient's values, and also help set realistic outcome expectations. The present methods and systems comprise a decision support tool which utilizes one or more predictive models to determines one or more risk scores and outputs the one or more risk scores. For example, the one or more risk scores may be associated with reamputation, mortality, and/or loss of mobility in patients undergoing an incident amputation at the Transmetatarsal (TM), Transtibial (TT) or Transfemoral (TF) level secondary to complications of diabetes and PAD. The present methods and systems determine one or more risk scores associated with a need for reamputation, a mortality risk, and the probability that a patient will achieve at least a basic level of ambulation. The present methods and systems provide an interactive decision support tool configured to determine and output the one or more risk scores thereby facilitating patients and surgeons making better informed decisions about risks during amputation-level shared decision-making discussions. Shared decision making at the time of amputation surgery may enhance post-amputation adjustment and recovery, survival, along with other key outcomes such as quality of life, mobility, and the risk of failure of amputation healing and need for additional surgery.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system;

FIG. 2 shows an example system;

FIG. 3 shows an example table;

FIGS. 4A-4B shows example tables;

FIG. 5 shows an example method;

FIGS. 6A-6F show example interfaces;

FIG. 7 shows an example interface;

FIG. 8 shows an example interface;

FIG. 9 shows an example system;

FIG. 10 shows an example diagram;

FIG. 11 shows an example diagram;

FIG. 12 shows an example table;

FIG. 13 shows an example table;

FIG. 14 shows an example table;

FIG. 15 shows an example index;

FIG. 16 shows an example chart;

FIG. 17 shows an example questionnaire;

FIG. 18 shows an example diagram;

FIG. 19 shows an example table;

FIGS. 20A-20C show example tables;

FIGS. 21A-21B show example tables;

FIG. 22 shows an example table;

FIGS. 23A-23B show example tables;

FIGS. 24A-24B show example tables and graphs;

FIG. 25 shows an example table;

FIG. 26 shows an example diagram;

FIGS. 27A-27B show an example table;

FIG. 28 shows an example table; and

FIGS. 29A-29B show an example graph and table.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for a decision support tool. The decision support tool may make use of statistical modeling and individual, patient specific predictor variables to determine at least one risk score and output the risk score for use by physicians and patients.

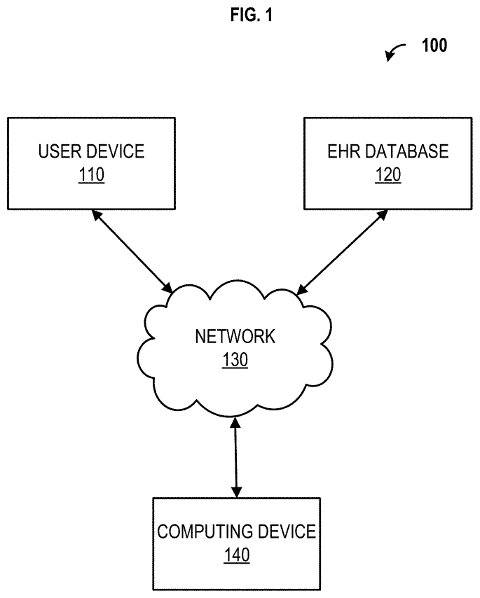

FIG. 1 shows an example system 100. The system 100 may comprise a user device 110, an EHR database 120, a network 130, and a computing device 140.

The user device 110, the EHR database 120, and the computing device 140 may be configured to send and receive data via the network 130. The network 130 may comprise any telecommunications network such as the Internet or a local area network. Other forms of communications can be used such as wired or wireless telecommunication channels, for example. The network 130 may be an optical fiber network, a coaxial cable network, a hybrid fiber-coaxial network, a wireless network, a satellite system, a direct broadcast system, an Ethernet network, a high-definition multimedia interface network, a Universal Serial Bus (USB) network, or any combination thereof.

The user device 110 may comprise, for example, a smartphone, a tablet, a computer, combinations thereof, and the like. The user device 110 may comprise an interface configured to receive an input from a user (e.g., a patient or a physician). For example, the user device may be configured to present, via a display, a fillable form. For example, if the fillable form may be associated with particular condition such as diabetes or hair loss while the supplemental patient data may comprise demographic data such as age, height, weight, region of residence, combinations thereof, and the like. The fillable form may comprise one or more selectable options. The fillable form may comprise, for example, a survey or questionnaire. For example, the fillable form may comprise a series of questions. The series of questions may be associated with a condition or diagnosis. For example, the series of questions may solicit, from a user, demographic data, comorbidity data, laboratory values and medications, health factors, combinations thereof, and the like. For example, demographic data may comprise data related to age, gender, income, educational attainment, marital status, race, combinations thereof and the like. For example, comorbidity data may comprise data related to diagnosis such as diabetes, congestive heart failure, previous amputation, a diagnosis of having chronic obstructive pulmonary disease (COPD), post-traumatic stress disorder (PTSD), anxiety, depression, combinations thereof, and the like. For example, laboratory values and medications may comprise metrics such as insulin levels, hormonal levels, platelet counts, white blood cell counts, combinations thereof, and the like. Health factors may comprise data related to height, weight, tobacco use, smoking history, alcohol use, functional status (e.g., does the patient live independently, assisted, or totally dependent?), combinations thereof, and the like.

The EHR database 120 may comprise a storage medium. As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof. The EHR database 120 may be configured to store one or more medical records. For example, the database 120 may comprise a VASQIP database. VASQIP is a surgical quality improvement data set developed to monitor the quality of surgical care in the Veterans Affairs (VA) Health Care System. Data in the database may be collected based on major operations within a healthcare system such as the VA. A person skilled in the art appreciate that the aforementioned is merely exemplary and not intended to be limiting to a particular database or health care system or provider. The EHR database 120 may comprise a VA CDW. The VA CDW may comprise inpatient and outpatient data, as well as demographic information. Data from the CDW may be used to determine whether the VASQIP surgical procedure was an incident amputation. The CDW may be configured to acquire additional predictor variables. The EHR database 120 may be configured to store supplemental patient data. For example, a medical record may comprise an identifier such as a name, address, telephone number, unique numerical identifier, combinations thereof, and the like. Supplemental patient data may comprise information associated with the patient, but not necessarily information included in or determined based on the fillable form. For example, the fillable form may be associated with particular condition such as diabetes, heart disease, previous amputation, or any other conditions.

The computing device 140 may be configured to execute one or more computing tasks. The computing device 140 may be configured to send and receive data. For example, the computing device may be configured to receive the patient data from the user device 110 and population data from the EHR database 120. The computing device may be configured to store and/or determine (e.g., select, generate, identify), one or more models. Each of the one or more models may be associated with one or more predictor variables. For example, the computing device may be configured to receive an input and apply it to one or more models. For example, the computing device 140 may be configured to receive the patient data and apply it the one or more models (e.g., the mobility model, the reamputation model, the mortality model, combinations thereof, and the like). The mobility model may be associated with population level mobility predictor variables. The reamputation model may be associated with population level reamputation predictor variables. The mortality model may be associated with population level mortality predictor variables. For example, the computing device 140 may be configured to receive the patient data and the population predictor variables and apply the patient data to one or more models. For example, the one or more models may comprise a mobility model, a reamputation model, a mortality model, combinations thereof, or the like.

The computing device 140 may be configured to determine one or more risk scores. Each risk scores of the one or more risk scores may be associated with a model of the one or more models. For example, a first risk score may be associated with a first model and a second risk score associated with a second model. For example. the computing device 140 may be configured to determine, by inputting patient data in to the mobility model, a mobility risk score. Likewise, the computing device 140 may be configured to determine, by inputting the patient data into the reamputation model a reamputation risk score. Similarly, the computing device 140 may be configured to determine, based on inputting the patient data into the mortality model, a mortality risk score. Regardless of the model, determining a respective risk score may comprise comparing the patient data to the population predictor variables and in particular, assigning coefficients to predictor variables that are common between the patient data and the population predictor variables. Based on identifying the population predictor variables, coefficients associated with the predictor variables may be determined. For example, in the population data, a variable such as smoking history may be strongly correlated with an indication of a reamputation after an incident (e.g., initial) amputation. By determining a smoking history associated with the received patient data, a determination may be made as to whether or not the present patient is likely to require reamputation. For example, a history of smoking may be associated with a smoking history coefficient on a per-model basis. That is to say, each model of the one or more models may be associated with a different value coefficient for smoking history. For example, in a first model (e.g., the mortality model), the smoking history predictor variable may be associated with a first value (e.g., +0.367) while an alcohol abuse predictor variable may be associated with a second value (e.g., +0.318). Meanwhile, in a second model (e.g., the reamputation model), the smoking history predictor variable may be associated with a third value (e.g., +0.418) while the alcohol abuse predictor variable may be associated with a fourth value (e.g., +0.521). Additional predictor variables and their associated coefficients are described in the Examples section of the present disclosure.

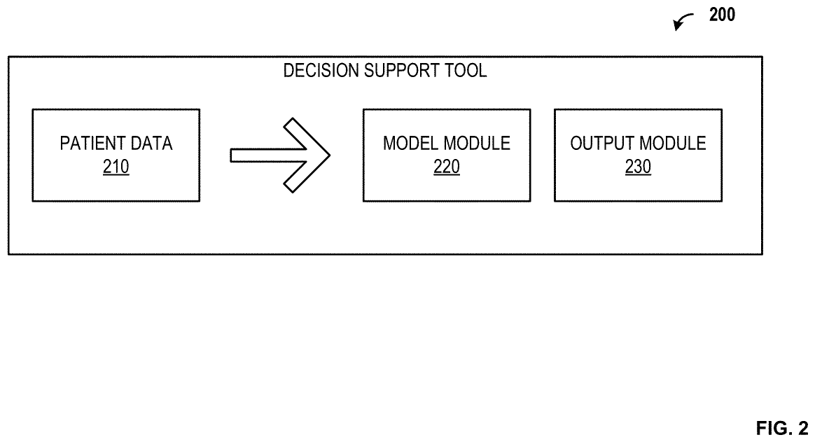

FIG. 2 shows an example decision support tool 200. The decision support tool may reside in any of the devices of FIG. 1 including the user device 110, the EHR database 120, or the computing device 140. The decision support tool 200 may be configured to receive patient data 210. For example, the DST 200 be configured to receive the patient data from the user device 110 and population data from the EHR database 120. The DST 200 may be configured to store and/or determine (e.g., select, generate, identify), the one or more models. For example, the one or more models may be stored in the model module 220. For example, the DST 200 be configured to receive an input and apply it to one or more models. For example, the DST 200 may be configured to receive the patient data and apply it the one or more models (e.g., the mobility model, the reamputation model, the mortality model, combinations thereof, and the like). For example, the DST 200 may be configured to receive the patient data 210 and the population predictor variables and apply the patient data to one or more models. For example, the one or more models may comprise a mobility model, a reamputation model, a mortality model, combinations thereof, or the like. Each of the one or more models may be associated with one or more predictor variables. For example, the mobility model may be associated with population level mobility predictor variables. For example, the reamputation model may be associated with population level reamputation predictor variables. For example, the mortality model may be associated with population level mortality predictor variables.

The model module 220 may determine a model and apply the received patient data to the model so as to determine the respective risk score. For example, applying the received patient data to the model may comprise comparing the received patient data to the one or more predictor variables associated with the model so as to determine a relationship (e.g., a correlation, a match, etc.) between the patient data and the one or more predictor variables.

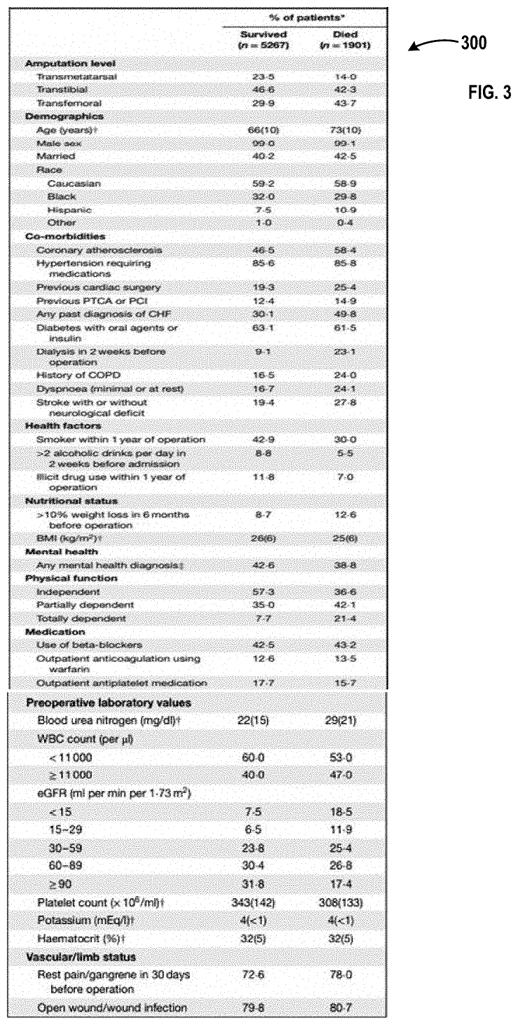

For each of the one or more models (e.g., the mobility model, the reamputation model, and the mortality model, each of the one or more predictor variables may be associated with a coefficient. For example, FIG. 3 shows example predictor variables 300. The predictor variables may comprise demographic indicators such as age, race, gender, or the like. The predictor variables may comprise comorbidities such as coronary atherosclerosis, hypertension, medical history indications, or diagnosis such as diabetes, for example. The aforementioned are merely exemplary and explanatory and are not meant to be limiting. The predictor variables may comprise health factors such as a history of smoking, drug use, diet and exercise and the like. The predictor variables may comprise nutritional information such as body mass index (BMI). The predictor variables may comprise an indication of physical function such as an independent status, a partially dependent status, or a totally dependent status, for example. The predictor variables may comprise medication history such as use of beta-blockers, anticoagulation, warfarin, or other medications. The predictor variables may comprise preoperative laboratory indicators such as blood urea nitrogen, white blood cell count, GFR, platelete count, potassium, hematocrit, and the like. The predictor variables may comprise an indication as to vascular and/or limb status such as rest pain/gangrene indications and/or open wound or wound infection indications.

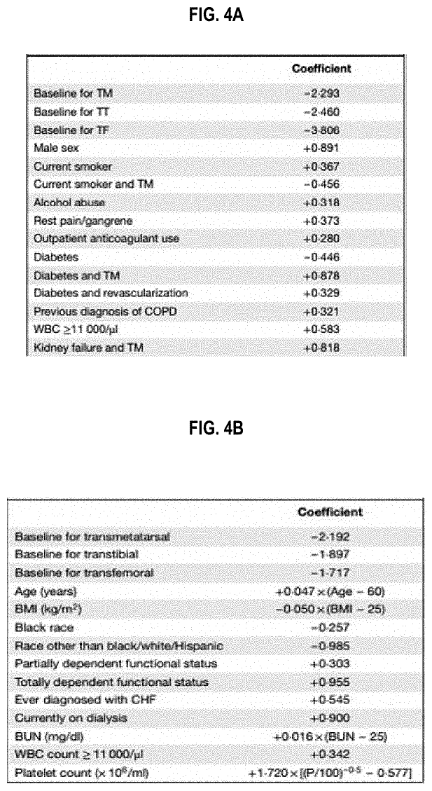

The example predictor variables 300 are associated with a risk of mortality, however, a person skilled in the art will appreciate that the predictor variables 300, combinations thereof, and the like, may be associated with mobility or reamputation as well. For example, FIG. 4A shows example coefficients associated with predictor variables of the reamputation model while FIG. 4B shows coefficients associated with predictor variables associated with the mortality model. Determining the coefficients associated with the mortality prediction model may comprise determining the observed or reported presence or absence of a predictor variable among individuals in population. The aforementioned coefficients and associated predictor variables are merely exemplary and explanatory and a person skilled in the art will appreciate that any coefficients and any predictor variables may be used.

The DST 200 may comprise an output module 230 configured to output the at least one risk score. For example, the output module 230 may be configured to send the at least one risk score to a remote device such, for example, the user device 110. Additionally and/or alternatively, the output module 230 may comprise a display (e.g., an interface) and the output module 230 may be configured to display the at least one risk score.

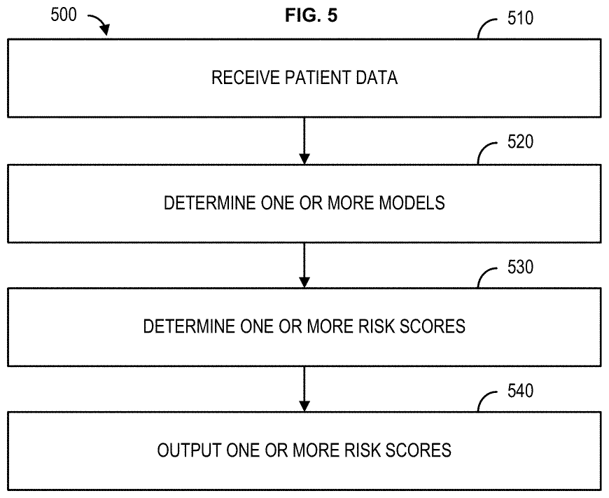

FIG. 5 shows an example method 500. The method 500 may be implemented on any one or any combination of the devices described herein. For example, the method 500 may be carried out by any of the devices of FIG. 1 including the user device 110, the EHR database 120, the computing device 140, combinations thereof, and the like. At step 410, patient data may be received. For example, the patient data may be initially received via a user interface of the user device 110. For example, the user device 110 may be configured to present, via a display, a fillable form. The fillable form may comprise one or more selectable options. The fillable form may comprise, for example, a survey or questionnaire. For example, the fillable form may comprise a series of questions. The series of questions may be associated with a condition or diagnosis. For example, the series of questions may solicit, from a user, demographic data, comorbidity data, laboratory values and medications, health factors, combinations thereof, and the like. For example, demographic data may comprise data related to age, gender, income, educational attainment, marital status, race, combinations thereof and the like. For example, comorbidity data may comprise data related to diagnosis such as diabetes, congestive heart failure, previous amputation, a diagnosis of having chronic obstructive pulmonary disease (COPD), post-traumatic stress disorder (PTSD), anxiety, depression, combinations thereof, and the like. For example, laboratory values and medications may comprise metrics such as insulin levels, hormonal levels, platelet counts, white blood cell counts, combinations thereof, and the like. Health factors may comprise data related to height, weight, tobacco use, smoking history, alcohol use, functional status (e.g., does the patient live independently, assisted, or totally dependent?), combinations thereof, and the like. The user device 110 may configured to send and receive data. For example, the user device 110 may be configured to send the patient data entered by the patient to the computing device 140. The computing device 140 may be configured to send and receive data. For example, the computing device 140 may be configured to receive the patient data from the user device 110.

At 520, one or more predictor models may be determined. The one or more predictor models may comprise a mobility model, a reamputation model, a mortality model, combinations thereof, and the like. Each model of the one or more models may comprise one or more predictor variables. For example, the mobility model may comprise mobility predictor variables, the reamputation model may comprise one or more reamputuation predictor variables, and the mortality model may comprise one or more mortality predictor variables. Each predictor variables of the predictor may, based on which model it is associated with, be associated with a predictor variable coefficient. For example, if a mobility predictor variable comprises an indication of whether or not the patient has undergone a previous amputation, previous amputation predictor variable may be associated with a first coefficient in the mobility model. However, the same predictor variable (e.g., whether or not the patient has undergone a previous amputation) may be featured in a different model (e.g., the reamputation model) and, when the previous amputation indicator is featured as a predictor variable in the reamputation model, it may be associated with a different coefficient.

At 530, one or more patient risk scores may be determined. The one or more patient risk scores may comprise an indication of how likely a patient is to suffer a particular symptom. For example, a first patient risk of the one or more patient risk scores may comprise an indication of how likely a patient is to suffer reduced mobility if they undergo an amputation. For example, a second patient risk score of the one or more patient risk scores may comprise an indication or how likely a patient is to require a reamputation (e.g., an additional amputation of the same limb albeit at a higher “level”). For example, a third risk score of the one or more patient risk scores may comprise an indication of how likely a patient is to die within one year of undergoing an incident amputation and/or a reamputation. Determining the one or more patient risk scores may comprise may comprise applying, on a per model basis, respective coefficients associated with the one or more predictor variables to the patient data. For example, if the received patient data comprises an indication that the patient is a former smoker, when applied to the mobility model, that indication may be associated with a first coefficient while an indication that the patient has undergone a previous amputation may be associated with a second coefficient. Similarly, when applied to the reamputation model, the indication that the patient is a former smoker may be associated with a third coefficient and the indication that the patient has previously undergone an amputation may be associated with a fourth coefficient.

At 540, the one or more patient risk scores may be output. Outputting the one or more patient risk scores may comprise sending, for example, to the user device 110 and/or some other computing device, or displaying by the computing device 140 via a display, the one or more patient risk scores.

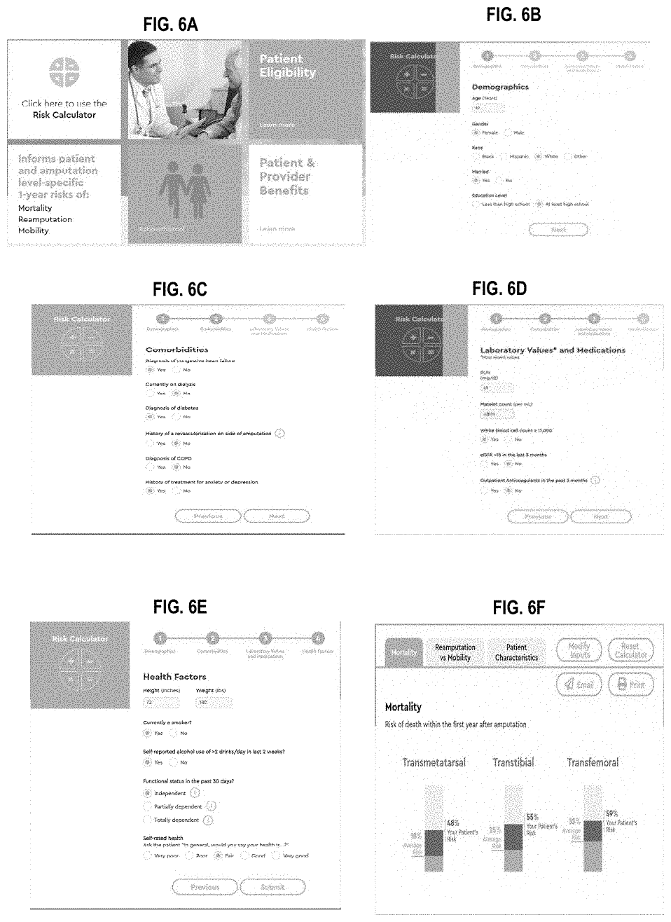

FIGS. 6A-6G show exemplary one or more user interfaces. The one or more user interfaces may be output (e.g., displayed) via a display device. The one or more user interfaces may be displayed on a display device configured to receive inputs. The one or more user interfaces may be configured to receive input. For example, the display device may comprise a touch-screen technology configured to convert a haptic input into a digital signal and thereby facilitate interaction with the one or more user interfaces. Additionally and/or alternatively, the display device may be configured to receive inputs via key-stroke, mouse indications and clicks, voice or other audio inputs and the like and combinations thereof. The one or more user interfaces may comprise various screens, subscreens, text boxes, selectable options, Tillable forms, combinations thereof, and the like. For example, as seen in FIG. 6A, the one or more user interfaces may be configured to display a navigable menu. The navigable menu may comprise selectable panes or images which may be configured to redirect the patient to a submenu. For example, as seen in FIG. 6B, the one or more user interfaces may comprise a user interface configured to receive demographic information. For example, as seen in FIG. 6C, the one or more user interfaces may be configured to receive information related to comorbidities. For example, as seen in FIG. 6D, the one or more user interfaces may be configured to receive information related to laboratory values and medications. For example, as seen in FIG. 6E, the one or more user interfaces may be configured to receive information associated with health factors. The one or more user interfaces may be configured to display the one or more risk scores. For example, FIG. 6F shows a display of one or more risk scores associated with mortality (e.g., a risk of death within the first year after amputation) broken down according to the location of the incident amputation (e.g., transmetatarsal, transtibial, transfemoral). The risk scores may be displayed in an easily understood format such bar graphs, pie charts, numerical values, or other visual representations.

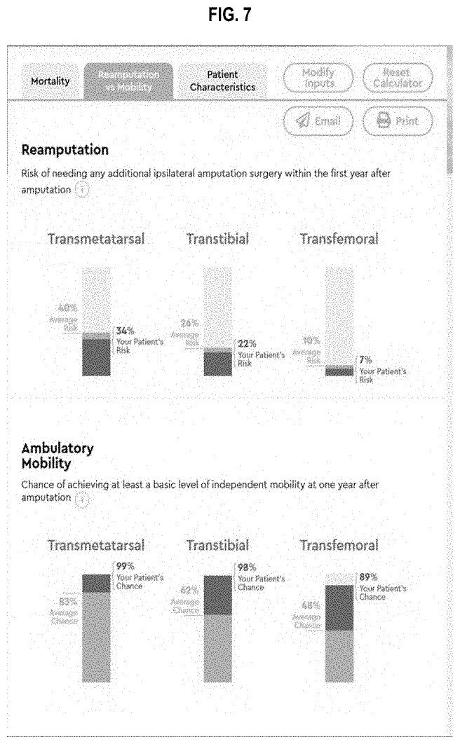

FIG. 7 shows an exemplary user interface configured to display the one or more risk scores. For example, FIG. 7 shows a display featuring risk scores associated with a risk of reamputation (e.g., a risk of needing any additional ipsilateral amputation surgery with in the first year after amputation) associated with different locations of incident amputation (e.g., transmetatarsal, transtibial, transfemoral). FIG. 7 also shows a display featuring risk scores associated with a risk of an increase or decrease in ambulatory mobility (e.g., chance of achieving at least a basic level of independent mobility at one year after amputation).

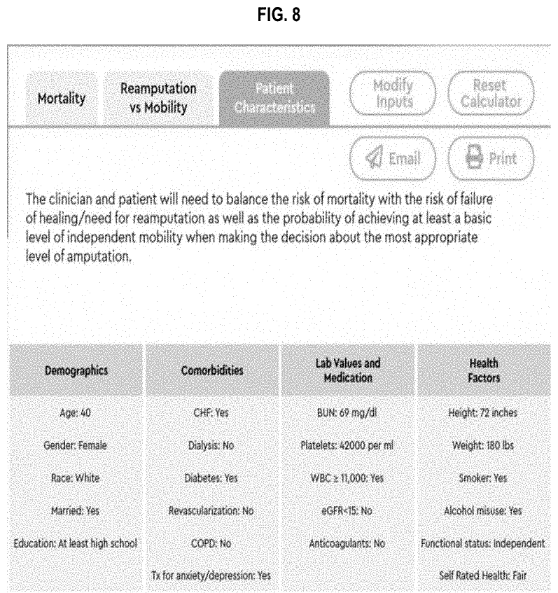

FIG. 8 shows an example display configured to display patient characteristics.

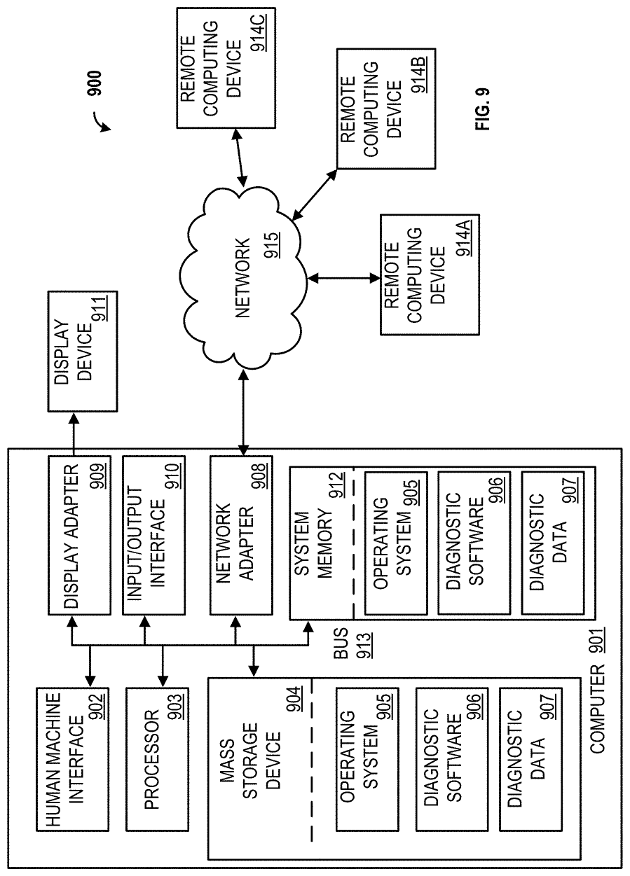

FIG. 9 shows a system 900 for error correction in accordance with the present description. The computer 901 may comprise one or more processors 903, a system memory 912, and a bus 913 that couples various system components including the one or more processors 903 to the system memory 912. In the case of multiple processors 903, the computer 901 may utilize parallel computing. The bus 913 is one or more of several possible types of bus structures, including a memory bus or memory controller, a peripheral bus, an accelerated graphics port, or local bus using any of a variety of bus architectures.

The computer 901 may operate on and/or comprise a variety of computer readable media (e.g., non-transitory media). The readable media may be any available media that is accessible by the computer 901 and may include both volatile and non-volatile media, removable and non-removable media. The system memory 912 has computer readable media in the form of volatile memory, such as random access memory (RAM), and/or non-volatile memory, such as read only memory (ROM). The system memory 912 may store data such as diagnostic data 907 and/or program modules such as the operating system 905 and diagnostic software 906 that are accessible to and/or are operated on by the one or more processors 903. The diagnostic data 907 may include, for example, one or more hardware parameters and/or usage parameters as described herein. The diagnostic software 906 may be used by the computer 901 to cause one or operations.

The computer 901 may also have other removable/non-removable, volatile/non-volatile computer storage media. FIG. 9 shows the mass storage device 904 which may provide non-volatile storage of computer code, computer readable instructions, data structures, program modules, and other data for the computer 901. The mass storage device 904 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 904, such as the operating system 905 and the diagnostic software 906. Each of the operating system 905 and the diagnostic software 906 (e.g., or some combination thereof) may have elements of the program modules and the diagnostic software 906. The diagnostic data 907 may also be stored on the mass storage device 907. The diagnostic data 907 may be stored in any of one or more databases known in the art. Such databases may be DB2®, Microsoft® Access, Microsoft® SQL Server, Oracle®, mySQL, PostgreSQL, and the like. The databases may be centralized or distributed across locations within the network 915.

A user may enter commands and information into the computer 901 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 903 via a human machine interface 902 that is coupled to the bus 913, but may be connected by other interface and bus structures, such as a parallel port, game port, an IEEE 1369 Port (also known as a Firewire port), a serial port, network adapter, and/or a universal serial bus (USB).

The display device 911 may also be connected to the bus 913 via an interface, such as the display adapter 906. It is contemplated that the computer 901 may have more than one display adapter 906 and the computer 901 may have more than one display device 911. The display device 911 may be a monitor, an LCD (Liquid Crystal Display), light emitting diode (LED) display, television, smart lens, smart glass, and/or a projector. In addition to the display device 911, other output peripheral devices may be components such as speakers (not shown) and a printer (not shown) which may be connected to the computer 901 via the Input/Output Interface 910. Any step and/or result of the methods may be output (or caused to be output) in any form to an output device. Such output may be any form of visual representation, including, but not limited to, textual, graphical, animation, audio, tactile, and the like. The display device 911 and computer 901 may be part of one device, or separate devices.

The computer 901 may operate in a networked environment using logical connections to one or more devices such a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device, and so on. Logical connections between the computer 901 one or more devices may be made via a network 915, such as a local area network (LAN) and/or a general wide area network (WAN). Such network connections may be through the network adapter 914. The network adapter 914 may be implemented in both wired and wireless environments. Such networking environments are conventional and commonplace in dwellings, offices, enterprise-wide computer networks, intranets, and the Internet.

Application programs and other executable program components such as the operating system 905 are shown herein as discrete blocks, although it is recognized that such programs and components reside at various times in different storage components of the computing device 901, and are executed by the one or more processors 903 of the computer. An implementation of the diagnostic software 906 may be stored on or sent across some form of computer readable media. Any of the described methods may be performed by processor-executable instructions embodied on computer readable media.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the methods and systems. Efforts have been made to ensure accuracy with respect to numbers (e.g., amounts, temperature, etc.), but some errors and deviations should be accounted for. Unless indicated otherwise, parts are parts by weight, temperature is in C or is at ambient temperature, and pressure is at or near atmospheric.

### Example 1

Methods

Study Design—Two multisite prospective cohort studies were conducted on individuals undergoing their first major LEA because of complications of PAD or diabetes. The first study was conducted between 2005 and 2009 at four sites: two Veterans Administration medical centers (located in Seattle and Denver), a Seattle-area university hospital, and a Seattle-based level I trauma center. The second study was conducted between 2010 and 2014 at four Veterans Administration medical centers (located in Seattle, Portland, Houston, and Dallas). To increase study power and to expand the generalizability of the model, both data sets were combined, ensuring a broad geographic and temporal range. Study operations and data elements collected were comparable for each study. The decision to perform TMA, BKA, or AKA was made at each site per usual care. Participants were assessed in-person or by telephone within 6 weeks after the definitive amputation procedure for baseline data and 12 months postsurgically. Additional data were gathered by systematic review of the medical records, and aspects of interview data were verified against the medical record. All assessments were performed by a trained study coordinator designated for each site. These studies were conducted in accordance with the procedures approved by human subjects review boards at each participating institution. All participants provided informed consent.

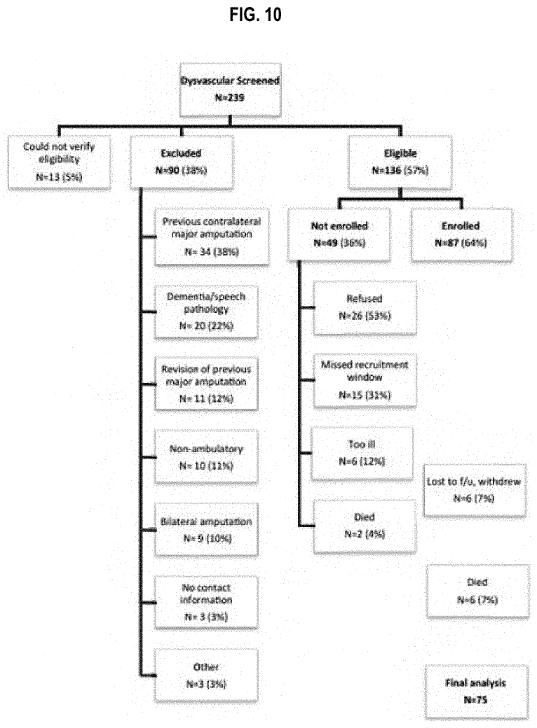

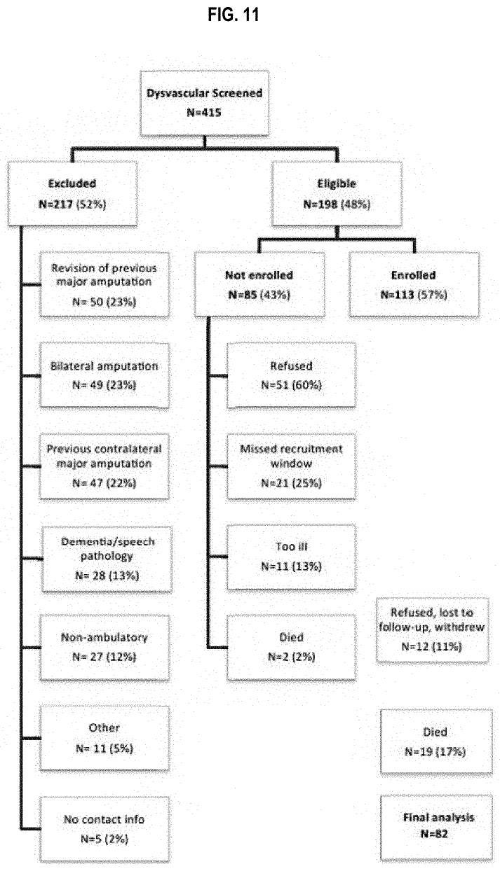

Participants—In the first prospective study, 239 potential participants were screened for participation. In the second prospective study, 415 potential participants were screened for participation. Participants were eligible if they were 18 years of age or older and (2) they were awaiting (or underwent in the last 6 weeks) a first major LEA (e.g., TMA, BKA, or AKA) related to complications of diabetes or PAD. Participants were excluded if (1) they had inadequate cognitive or language function to consent or to participate defined by more than four errors on the Short Portable Mental Status Questionnaire or they were nonambulatory before the amputation for reasons unrelated to PAD or diabetes. Among the potential participants in the first study, 136 (57%) met study criteria; 87 participants (64% of eligible) agreed and were able to participate (FIG. 10). Among the potential participants in the second study, 198 (48%) met study criteria; 113 subjects (57% of eligible) agreed and were able to participate (FIG. 11). A total of 200 participants made up the combined baseline study population.

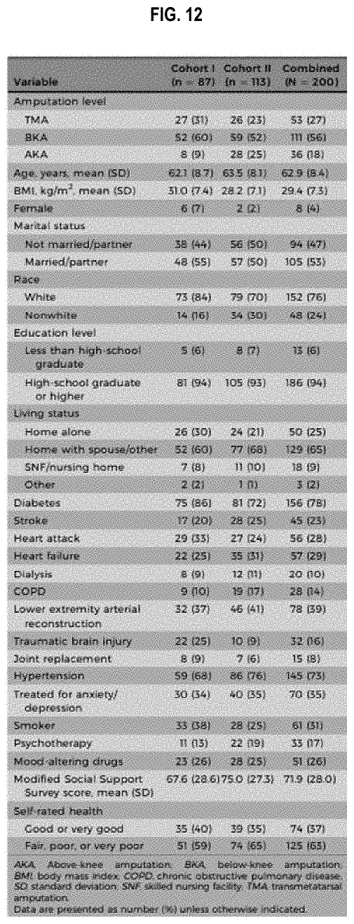

Predictor Variables—Predictors were chosen on the basis of three main criteria: (1) clinical expert consensus on predictive importance of specific variables; (2) literature support for the predictive importance of specific variables; and (3) they could be easily obtained before amputation in the clinical/surgical setting. Baseline measures included age, gender, marital status, race (self-reported and coded as white or nonwhite because of very low proportion of nonwhite), education level, living environment, body mass index (BMI), self-rated health, tobacco use, several comorbid medical conditions, history of anxiety or depression, and level of amputation. The anatomic level of amputation (ie, TMA, BKA, or AKA) was determined from the medical record, as was the primary etiology (diabetes vs PAD). The presence or absence of the following specific comorbid conditions or procedures was self-reported and then verified in the medical record: diabetes, previous lower extremity arterial reconstruction, traumatic brain injury, hypertension, joint replacement, chronic obstructive pulmonary disease (COPD) currently on dialysis, previous heart attack, heart failure, and stroke. If the condition was not reported but identified in the medical record, the participants were counted as having the condition. If the condition was self-reported but not identified in the record, the participants were counted as having the condition. We also asked participants whether they had participated in individual or group psychotherapy, whether they were taking medications for mood, and whether they had a history of treatment for anxiety or depression. We assessed the degree of social support using the brief version of the Modified Social Support Survey, a measure of perceived social support developed initially as part of the Medical Outcomes Study and subsequently shortened (to 5 items from 18) as part of the Multiple Sclerosis Quality of Life Inventory. Possible total scores range from 0 to 100, with higher scores indicating greater perceived social support. Participants were considered smokers if they endorsed smoking “every day” or “some days” before amputation and nonsmokers if they endorsed “not smoke at all.” All baseline assessment measures are presented in FIG. 12.

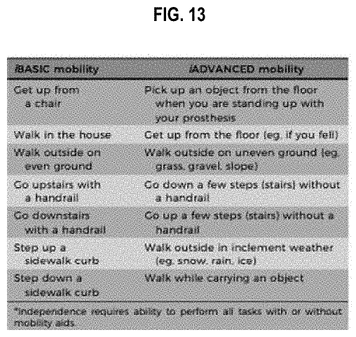

Primary Outcome Measure: Locomotor Capabilities Index 5-Level (LCI-5 scale).—Mobility was assessed using the LCI-5 at 12-month follow-up; 14-items are graded on a 5-level ordinal scale ranging from “unable to perform the activity” (0 points) to “able to perform independently without assistance” (4 points).12 Possible scores for the LCI-5 range from 0 to 56 points, with higher scores representing higher function. Among amputees, the LCI-5 has well-established internal consistency, test-retest reliability, and content, discriminant, and criterion validity. The subscales were generated from the measure (FIG. 13), namely, independent (i) in basic (iBASIC) mobility (seven basic items) and independent (i) in advanced (iADVANCED) mobility (seven advanced items). iBASIC mobility or iADVANCED mobility was achieved if a participant was able to perform all of the tasks associated with the subscale independently with or without ambulatory aids. These were the two primary outcomes for our prediction models. All but one individual who achieved iADVANCED also achieved iBASIC mobility. This individual was independent without the use of an assistive device for six of seven basic mobility elements (the exception was that the person required assistance for stepping down a sidewalk curb).

Statistical Analysis—All predictors considered for inclusion in the development models and their format and categorization are presented in FIG. 12. Age and BMI were centered (at 60 years and 30 kg/m², respectively) to aid in the interpretation of the model coefficients. In modeling the association with mobility outcomes, we also considered quadratic terms in age and BMI to accommodate possible nonlinear relationships. Although we recognize the potential for factors such as patient age, BMI, marital status, and presence of COPD to modify the impact of amputation level on mobility outcomes, because of sample size constraints, especially in the AKA group, we did not consider inter-action terms in the primary models. In fact, the optimism-adjusted area under the curve estimates were lower when interaction terms were included. The main effects of amputation level were forced to be retained. Other variables were retained with a P value of 0.20. To quantify the discrimination of each model, we estimated the C statistic and the discrimination slope. Calibration was assessed using the Hosmer-Lemeshow (H-L) goodness-of-fit test and plots of the observed proportions against estimated probabilities using a Lowess smoothing curve for visualization. Outliers in the box plots of predicted probabilities were inspected for clinical plausibility. The developed models were internally validated with bootstrap sampling to obtain estimates of the optimism of the C statistic and the difference in predicted probabilities for those who did and did not achieve iBASIC and iADVANCED mobility (ie, the discrimination slope). Bootstrap samples were drawn with replacement and with the same size as the original sample. Model selection was carried out for each bootstrap sample and model performance assessment compared with that on the original sample. This was repeated 500 times to obtain stable estimates of the average optimism of the C statistic and discrimination slope for each model.

To demonstrate the clinical utility of AMPREDICT-Mobility, the estimated probabilities (and associated 95% prediction intervals) of achieving iBASIC and iADVANCED mobility at 1 year after amputation were considered in hypothetical clinical scenarios and included in the Appendix (online only). Statistical analyses were performed using Stata 9.0.

Results

Baseline Characteristics—Among the 87 participants enrolled in the first cohort, 4 participants (5%) formally withdrew, 2 (2%) were lost to follow-up, and 6 (7%) died during the 12-month follow-up period; 75 participants completed their 12-month interview (86%; FIG. 10). Among the 113 subjects enrolled in the second cohort, 5 subjects (4%) formally withdrew during the course of the study, 1 subject (w 1%) refused the 12-month interview, 6 (5%) were lost to follow-up, and 19 subjects (17%) died during the 12-month follow-up period; 82 subjects (73%) completed their 12-month interview (FIG. 11). FIG. 12 summarizes the baseline characteristics of both cohorts. In total, 157 subjects (79%) completed their 12-month follow-up and were included in the two prediction models.

LCI-5 scores and achievement of iBASIC and Iadvanced mobility—The mean LCI-5 score at 12-month follow-up was 36.1 (standard deviation, 17.1; range, 0-56). Among the 157 subjects in the combined sample who completed their 12-month follow-up, 54 (34%) did not achieve iBASIC mobility; 103 (66%) achieved iBASIC mobility, and of these, 51 (32%) also achieved iADVANCED mobility. Differences in achieving iBASIC mobility by amputation level were statistically significant (C², P=0.007), with 83%, 62%, and 48% of TMA, BKA, and AKA amputees achieving this level of mobility. A statistically significant difference across amputation levels was not observed in those achieving iADVANCED mobility, with 39%, 33%, and 20% of TMA, BKA, and AKA amputees achieving that level (C², P=0.26).

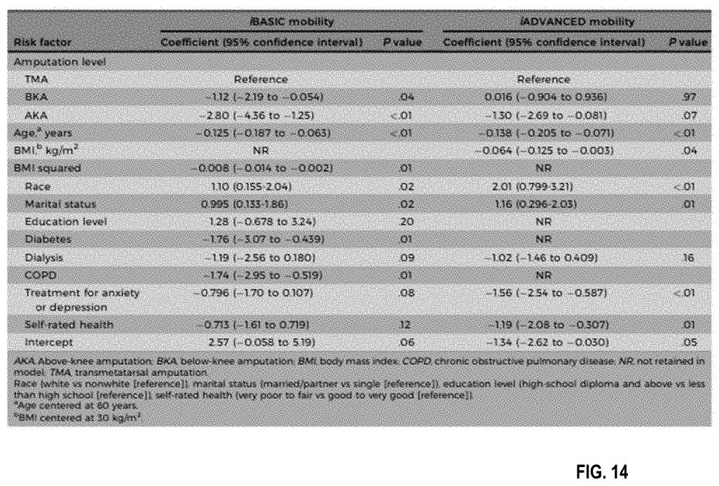

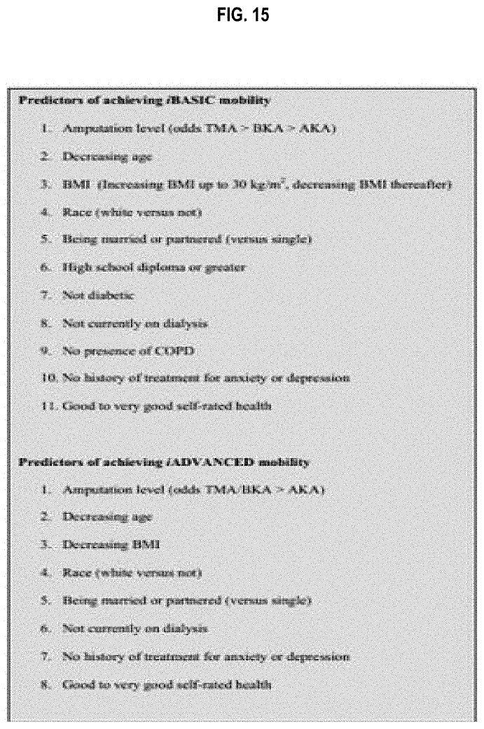

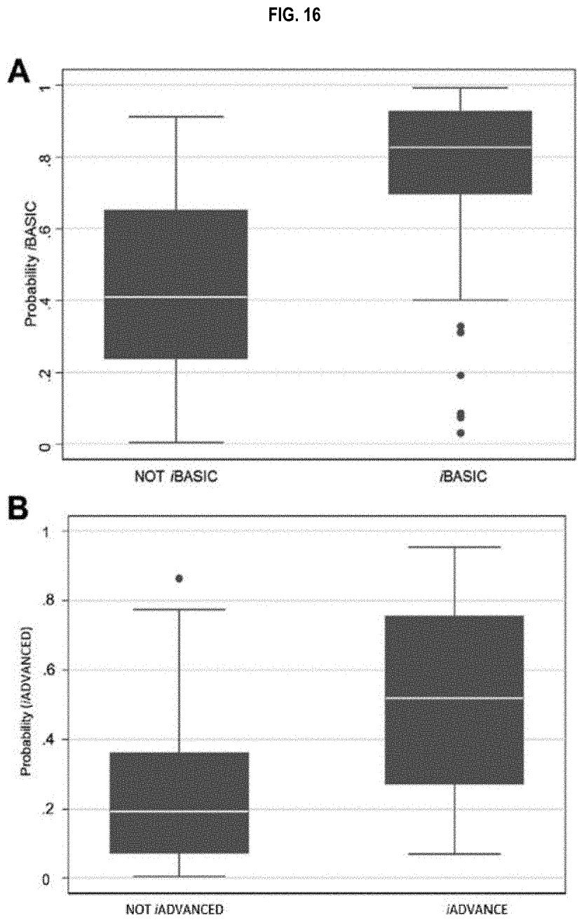

Prediction Model Development—The selected logistic regression models for iBASIC and iADVANCED mobility with regression coefficients are presented in FIG. 14, and the variables retained in the final models are listed in FIG. 15. Predictive factors associated with reduced odds of achieving iBASIC mobility were increasing age, COPD, dialysis, diabetes, prior history of treatment for depression or anxiety, and very poor to fair self-rated health. Those who were white, were married, and had at least a high-school degree had a higher probability of achieving iBASIC mobility. The odds of achieving iBASIC mobility increased with increasing BMI up to 30 kg/m² and decreased with increasing BMI thereafter. In secondary analyses, we considered including in the prediction model selected interaction terms for amputation level with age, BMI, marital status, and presence of COPD. However, the interaction terms either were not selected or were in directions that were contrary to our under-standing of the roles of these variables. In addition, there was little gain in predictive value when the interaction terms were included. The estimated C statistic was 0.85, and the H-L goodness-of-fit test indicated adequate calibration (P=0.07). The predicted probabilities for those who did and did not achieve iBASIC mobility are illustrated in FIG. 16A, and show good separation of the two groups. The difference in mean predicted probability was 33% and the difference in medians was >40%, demonstrating good discrimination. Whereas 75% of subjects who achieved iBASIC mobility had estimated probabilities >70%, we observed seven outliers (6.8% of subjects who achieved this level of mobility) who had a probability of <40% for achieving iBASIC mobility and yet successfully achieved it. The plot of predicted vs observed probabilities indicated good fit of the model. The prediction model for iADVANCED mobility included the same predictors as the model for iBASIC mobility with the exception of diabetes, COPD, and education level. Education level did not have a strong association with iBASIC mobility (P=0.2 in the final prediction model), and COPD came close to inclusion in the iADVANCED model (P=0.2005). The C statistic was 0.82, and the H-L goodness-of-fit test indicated good calibration (P=0.49). The predicted probabilities for those who did and did not achieve iADVANCED mobility are illustrated in FIG. 16B and show good separation of the two groups. The difference in mean predicted probability was 29% and the difference in medians was >30%, demonstrating good discrimination. We observed one outlier who had a probability of 86% for achieving iADVANCED mobility and failed to do so. The plot of predicted vs observed probabilities demonstrated good model fit.

Prediction Model Validation—The bootstrapping procedure provided estimates of the optimism of the estimated C statistic and discrimination slope of each model. Bootstrap estimates of the optimism for the C statistic were 0.11 for both iBASIC and iADVANCED mobility models and for the discrimination slope 0.14 and 0.13, respectively. This demonstrated some overoptimism of the original model development, with the optimism-adjusted C statistic for iBASIC and iADVANCED mobility being 0.74 and 0.71, respectively, and the discrimination slope 19% and 16%, respectively.

Discussion

The primary goal of this investigation was to develop and internally validate a set of mobility prediction models for use among patients with first major dysvascu-lar LEA (AMPREDICT-Mobility) that uses baseline patient factors, including amputation level, to predict iBASIC mobility and iADVANCED mobility 12 months after dys-vascular LEA.

Prediction modeling is currently being used in many as-pects of medicine, including cancer care, the evaluation of risk of death after myocardial infarction, diabetes care, and spinal cord injury. The current movement in health care toward shared decision-making requires not only general population evidence but evidence that supports individual probabilities of risks and benefits. AMPREDICT-Mobility uses two prediction models that enable the prediction of probable independence in all mobility subtasks included in iBASIC and iADVANCED. The prediction models are patient specific and use easily obtainable preamputation variables. There are no existing predictive models of mobility outcome after amputations that allow comparison with AMPREDICT-Mobility. However, previously published retrospective and cross-sectional studies have demonstrated increasing age associated with adverse functional and mobility outcomes. Anxiety and depression are common after amputation and can adversely affect quality of life. Some studies suggest that there is no relationship between depression and prosthetic use, whereas others have found that depression was predictive of poorer mobility outcomes. These studies describe the association between anxiety/depression after amputation and postamputation outcomes, whereas the current predictive model uses pre-existing anxiety and depression. Self-rated health has not been examined in amputee outcomes. It is a complex multidimensional measure that has many underlying determinants that may vary by study population. The validity of self-rated health and its contribution to the prediction of mobility outcome in amputees are reflected by its association with disability, health care utilization, and mortality. Dialysis has been associated with lower functional outcome scores and reduced prosthetic use. The effect of BMI on amputee mobility outcome is controversial. Kalbaugh et al found no effect on mobility, whereas Rosenberg et al did find reduced prosthetic use with increased BMI. The effect of BMI on probable mobility outcome in the two prediction models reflects some of the differing results seen in the literature. In the iBASIC model, a quadratic effect of BMI was associated with an increased probability of iBASIC mobility with increasing BMI up to 30 kg/m2 and decreased probability thereafter; in the iADVANCED model, increasing BMI reduced the probability of independence over the entire range of BMI. Racial factors and mobility outcome after amputation have not been evaluated in the literature; however, African American racial background has been associated with increasing rates of amputation, reduced survival, and increased odds of having a higher level of amputation. Similarly, the effect of marital status has not been studied, although social integration, which may be a surrogate for marital status, has been associated with improved function.

It is important to consider not only the predictors that were incorporated into the predictive model but also the potential predictors that were not included. This study was unique in that baseline perioperative variables also included key individual medical comorbidities, smoking, social support, psychotherapy, treatment for mental health disorders, and revascularization surgery and joint arthroplasty. Perhaps surprisingly, comorbid medical conditions such as prior myocardial infarction, diagnosis of congestive heart failure, and prior stroke were not retained in the models. Intuitively, one would consider these factors influential in mobility outcome; however, a prior systematic review of the literature also did not support these associations.

The inclusion of amputation level in the models allows the clinician and patient to obtain a probability of achieving/BASIC and ADVANCED mobility at each major level. Interestingly, amputation level had a large effect on achieving/BASIC mobility. Amputation at the BKA and AKA levels compared with the TMA level had an adverse impact on the probability of achieving/BASIC mobility. The BKA level compared with TMA had little effect on achieving/ADVANCED mobility, whereas the AKA level had an adverse effect.

It is well known that significant associations with an outcome are not sufficient to ensure accurate prediction. Although our prediction models for iBASIC and iADVANCED mobility show good discrimination and calibration, the predicted probabilities for some covariate patterns have wide prediction intervals (see case studies in Appendix, online only). Despite this being the largest prospectively enrolled study of dysvascular amputees with 12-month longitudinal follow-up, the sample size was modest and contributed to the relatively wide prediction intervals. Nevertheless, the prediction models do provide a common language for communication of anticipated mobility after amputation and provide useful evidence on expected mobility to inform patients and providers. Although we have adjusted for optimism in assessing model performance by internal validation, ideally these models should be externally validated in the future with larger sample sizes.

Our predictive model was developed using the assessment of self-rated health in the immediate postoperative period; therefore, it may not reflect the self-rated health during the immediate preoperative period, when the prediction model would be used. However, participants were asked to recall their self-rated health before the amputation. Although we have not established the validity of this method, prior published research indicates that the proportion of individuals with diabetes who report fair, poor, and very poor ratings of health is similar. Furthermore, prior research does indicate that during a hospitalization for an acute medical event, the recall of self-rated health before the event is still predictive of key outcomes. Whereas the LCI can be divided into a basic and advanced scale, the basic scale does not include very basic mobility elements, such as bed and toilet transfers or wheeled mobility. Therefore, in counseling a patient with the AMPREDICT-Mobility model, it will be important that it be done with a full knowledge of what mobility activities are being predicted.

Finally, the iBASIC prediction model had seven outliers. Of the 103 subjects who achieved iBASIC mobility, these subjects were predicted not to achieve this and did achieve it. Examination of patient characteristics did not reveal a defined pattern to explain this finding. The majority of these participants were diabetic, were not married, and rated their health fair to poor. The effects of these factors are complex and multidimensional; therefore, their effect in different individuals may vary.

### CONCLUSION

The absence of prediction models has contributed to the challenges that medical providers face in communicating the risks and benefits of different amputation levels on anticipated mobility outcome. AMPREDICT-Mobility is a novel predictive tool that was built on a wide spectrum of biopsychosocial factors existing at the time of amputation surgical decision-making. It is designed to quantify the probability that either iBASIC or iADVANCED mobility will be achieved, depending on the amputation level, to inform communication between the patient and surgeon during the preoperative period. Future application may involve an on-line calculator or smart phone application that can be used in the clinical environment.

### Example 2

Methods

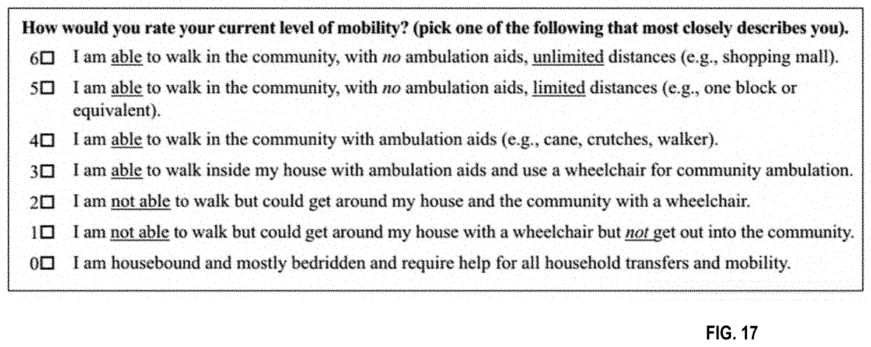

Study Design—This study was part of a larger multisite prospective cohort study of individuals undergoing major unilateral lower extremity amputation (transmetatarsal, transtibial, transfemoral) secondary to peripheral arterial disease or diabetes at four Veteran's Administration Medical Centers. Participants were assessed via in-person or telephone interview at baseline (i.e. within seven days of the definitive amputation procedure), six weeks, four months, and 12 months postsurgically. FIG. 17 shows an example evaluation questionnaire. Local institutional review boards approved study procedures. Informed consent was obtained from all individual participants included in the study. All assessments were performed by a trained study coordinator designated for each site that was responsible for recruitment, interviews, completion of case report forms, and routine monitoring of enrolled patients.

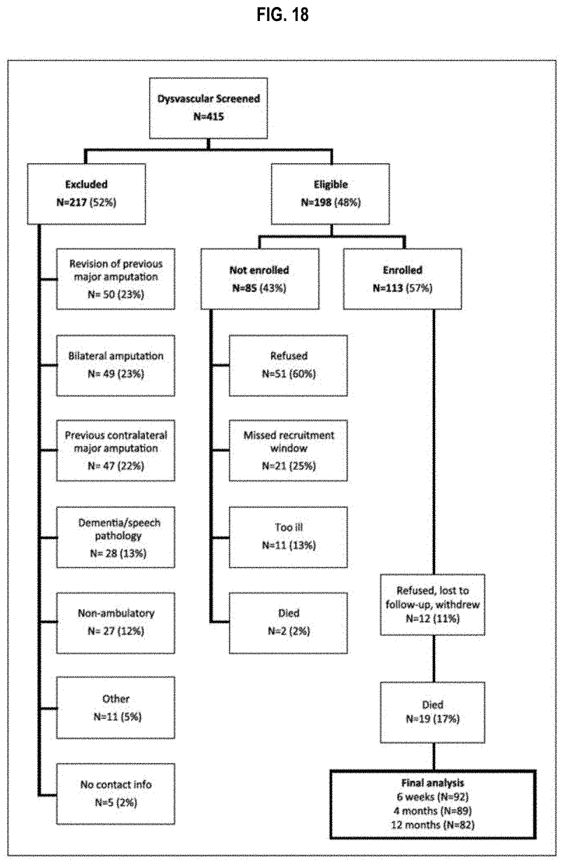

Participants—Potential subjects were screened in person or in the medical record before being approached for consent between August 2010 and April 2013. Subjects were considered eligible if they were 18 years or older, were awaiting (or underwent in the last seven days) a first major lower extremity amputation, and the primary cause of amputation was com-plications of diabetes or peripheral arterial disease. Subjects were excluded if they had inadequate cognitive or language function to consent or participate, defined by more than four errors on the Short Portable Mental Status Questionnaire, or were non-ambulatory before the amputation for reasons unrelated to peripheral arterial disease or diabetes. Of 415 individuals screened, 198 (48%) met study criteria; 85 (43% of eligible) refused, missed the recruitment window, or died before they could be enrolled; and 113 (57%) participated in the study (FIG. 18). Among the 217 who were ineligible, the most common reasons were prior amputation (23% revision surgeries, 22% prior contralateral amputation) or bilateral amputation (23%).

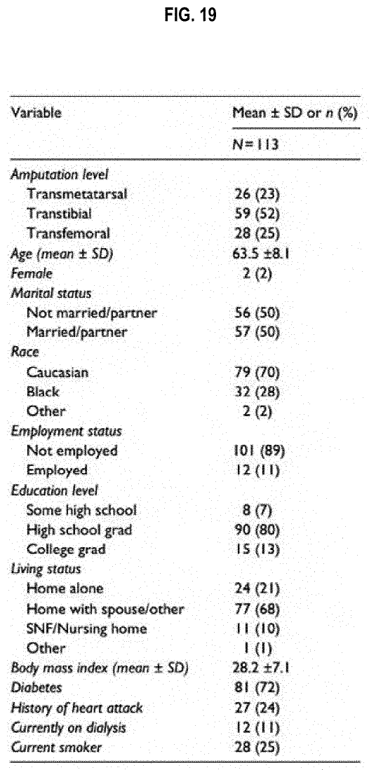

Baseline Assessment Measures—Baseline measures included socio-demographics, smoking status, and common comorbid medical conditions. The primary etiology for amputation was categorized as diabetes or peripheral artery disease, and the anatomic level of amputation was categorized as transmetatarsal, transtibial, or trans-femoral (FIG. 19).

Data Analysis—See FIG. 19 for descriptive statistics of presurgical variables. While the AMPSIMM can be conceptualized as an ordinal scale, and used in this way to characterize individual amputees for the purposes of establishing validity and for evaluating the effects of interventions on a population of amputees, the AMPSIMM was tested as both a categorical and a continuous variable for statistical purposes. To justify this, we assessed the assumption of normality of the AMPSIMM using the Shapiro—Wilk test. The AMPSIMM was found to display a normal distribution at six weeks, four months, and 12 months (p=0.10, 0.57, and 0.98, respectively). Non-parametric statistics were employed when evaluating correlations of the AMPSIMM and other measures. The Chi-square test for trend was used when evaluating the ordered AMPSIMM categories by amputation level. Stata 9.1 was used for the statistical analyses described.

Criterion Validity—The Locomotor Capabilities Index-5 was chosen as the reference standard for the measurement of concurrent and predictive criterion validity because it assessed the degree to which ambulation aids were used and covered a relevant range of mobility tasks. In addition to having a relevant range of content, this measure has well-established internal consistency, test—retest reliability, and validity (content, discriminant, and criterion).14,20-23 The Locomotor Capabilities Index-5 was administered at six weeks, four months, and 12 months after amputation.

To evaluate the concurrent criterion validity of the AMPSIMM, the Spearman's rank correlation coefficient was used to determine the correlation between the AMPSIMM score and the Locomotor Capabilities Index-5 score at six-week, 4-month, and 12-month follow-ups. Correlations of 0.1 were considered “small,” 0.3 as “medium,” and 0.5 as “large.” To evaluate the predictive criterion validity of the AMPSIMM, we evaluated the association of the six-week and four-month AMPSIMM scores with the 12-month Locomotor Capabilities Index-5 score using the Spearman's rank correlation coefficient. By ensuring that the AMPSIMM scores preceded the reference standard assessment chronologically, this was considered an assessment of predictive validity.

Construct Validity—Construct validity represents a quantitative form of assessing validity by selecting other measures that evaluate the same or similar constructs and hypothesizing a priori the strength of the correlation. Hours of prosthetic use was measured among individuals who had been fitted with a prosthesis by asking “On average, how many hours per day are you walking with your prosthesis.” Functional restriction was assessed using the functional restriction subscale scores of the Trinity Amputation and Prosthesis Experience Scales (TAPES).26 The TAPES include nine sub-scales, measuring psychosocial outcomes, activity restriction, prosthetic satisfaction, pain, and general health. The activity restriction subscale is further divided into an athletic activity restriction, functional restriction, and social restriction—the higher the score, the higher the restriction with scores ranging from 0 to 8. The functional restriction subscale was selected a priori because the items were most relevant to a dysvascular amputee population and represented the conceptual inverse of the function domain that the AMPSIMM measures.

Satisfaction with mobility was assessed at four months and 12 months after amputation with a single item measure developed in a prior study. Subjects responded to the question: “How satisfied are you with your current walking ability?” using a 10-point Likert scale, where 0 represented “not at all satisfied” and 10 “extremely satisfied. This scale was also dichotomized establishing a subject as “satisfied” with a score of 6 to 10 and “not satisfied” with a score of 0 to 5.

To evaluate one form of convergent construct validity, the association between the four-month and 12-month AMPSIMM scores and four-month and 12-month hours of daily prosthetic use, TAPES functional restriction score, and satisfaction with mobility scores were evaluated using Spearman's rank correlation coefficients. It was hypothesized that the strongest correlation would be with hours of prosthetic use, followed by activity restriction (using the TAPES), and then satisfaction with mobility. Non-parametric tests for trend were performed with cross tabulations of the AMPSIMM score and those “satisfied” or “not satisfied” with mobility to assess whether there was a trend in the ordering of the AMPSIMM scores by those “satisfied” vs. “not satisfied.”

To evaluate “known group” validity, mean AMPSIMM scores were compared by anatomic amputation level, hypothesizing that transmetatarsal amputees would have higher mean scores, followed by transtibial amputees, and transfemoral amputees. Cross tabulations were performed using the AMPSIMM response options by amputation level; the same non-parametric test for trend was used to assess whether there was a trend in the ordering of the AMPSIMM score by amputation level.

Responsiveness—As the mobility of a new amputee typically improves during the first year post-amputation, it was hypothesized that the AMPSIMM scores would also improve over time. Several outcomes studies have used different methods to estimate magnitude of change over time in terms of an effect size. Some report that there is no definitive evidence that any method offers specific advantages. Therefore, to evaluate responsiveness, the change score between the six-week and 12-month assessments was calculated and divided by the standard deviation of the AMPSIMM's change score to derive the standardized response mean. The change score standard deviation was imputed by using a formula recommended by the Cochrane collaboration. Using Cohen's effect size criteria (not to be confused with the previous criteria for correlations),24 0.2 to 0.49 was considered a “small” effect, 0.5 to 0.79 a “moderate” effect, and 0.8 to infinity, a “large” effect. To assess the floor and ceiling effects of the AMPSIMM score, the percentage of subjects who achieved the minimum and maximum score was computed. Percentages greater than 15% were considered as demonstrating a floor or ceiling effect.

Results

Baseline Characteristics—The majority of the 113 subjects enrolled in the study had transtibial amputations (52%) followed by transfemoral (25%) and transmetatarsal level (23%) amputations (FIG. 19). Differences between baseline variables, comparing all subjects to those that completed each follow-up, were small and not statistically or clinically significant.

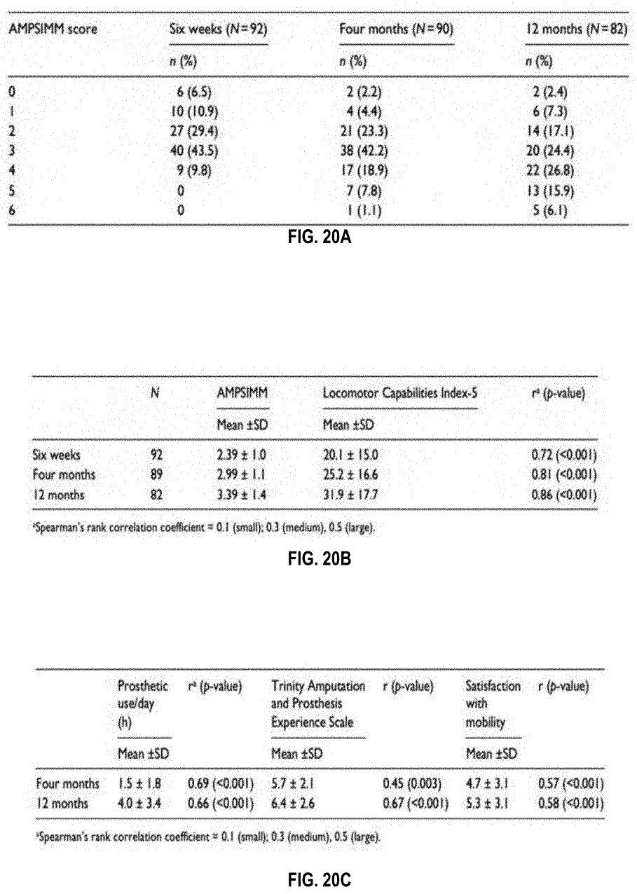

Despite quantitative evidence for normality at all time points, raw AMPSIMM scores were more heavily distributed in the lower region at six weeks, with no subjects achieving a score of five or six (FIG. 20A). This was expected, since most patients are very early in their rehabilitation process and therefore higher scores are not expected. The distribution became more evenly distributed at subsequent follow-up—especially at 12 months.

Criterion Validity (Concurrent)—The mean AMPSIMM and Locomotor Capabilities Index-5 scores at six weeks, four months, and 12 months are presented in FIG. 20B. The AMPSIMM demonstrated “large” correlations with the Locomotor Capabilities Index-5 scores at all follow-up times. The strength of the correlation increased with each subsequent follow-up and the relationship appeared linear by visual inspection.

Criterion Validity (Predictive)—The correlation between the six-week AMPSIMM score and the 12-month Locomotor Capabilities Index-5 score was computed. This relationship was considered less than “small” and not statistically significant (r=0.07; p=0.56). The correlation between the four-month AMPSIMM score and the 12-month Locomotor Capabilities Index-5 score was considered “medium” and statistically significant (r=0.40; p=0.004). This suggests assessing the AMPSIMM at four months has some predictive qualities, but not at six weeks.

Construct Validity: Hours of Prosthetic Use—Among those who had been fitted with a prosthesis (n=26 and 47, at four months and 12 months, respectively), the mean hours of prosthetic use are presented in FIG. 20C. The correlation between the AMPSIMM score and hours of prosthetic use at these time points were considered “large” correlations.

Construct Validity: TAPES—The mean TAPES functional restriction scale scores at four months and 12 months are presented in FIG. 20C. The correlations between the AMPSIMM score and the TAPES functional restriction score at these time points were “medium” and “large,” respectively.

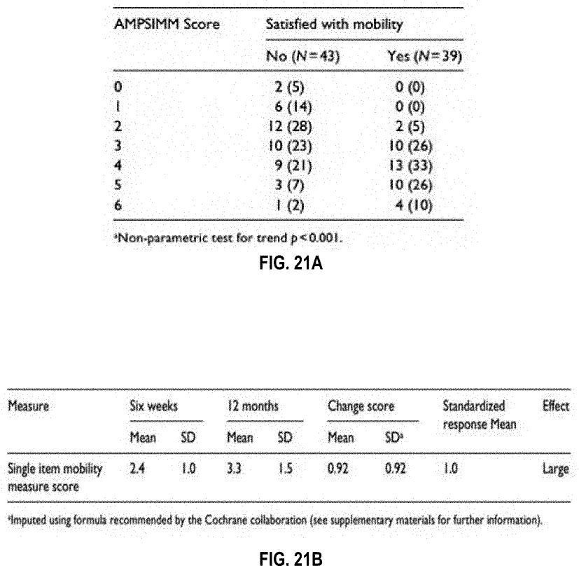

Construct Validity: Satisfaction with Mobility—The mean satisfaction with mobility scores at four months and 12 months are presented in FIG. 20C. The correlations were considered “large” at both time points. Further, those “satisfied” with their mobility were significantly more likely to have a higher AMPSIMM score (test for trend p<0.001) (FIG. 21A).

Construct Validity: Known Group—The mean 12-month AMPSIMM scores differed among amputation levels as hypothesized. AMPSIMM scores were highest for transmetatarsal amputees and lowest for transfemoral amputees (means for transmetatarsal, transtibial, and transfemoral amputees were 4.2, 3.2, and 2.9, respectively).

Responsiveness—When measuring the change in score from six weeks to 12 months after amputation, the AMPSIMM score improved significantly (mean change 2.4) with a standardized response mean of 1.0, representing a “large” effect FIG. 21B.

Discussion

Psychometric evaluation of the AMPSIMM supports the utility of this measure to quantify mobility in the dysvascular amputee population. AMPSIMM has moderate to strong criterion and construct validity, as well as excellent responsiveness and no indication of floor/ceiling effects. Although it was not designed to replace existing measures, the AMPSIMM is unique in terms of its brevity, ease of administration, utility in quantifying mobility across the rehabilitation continuum in the dysvascular amputee population, and ability to define mobility in clinically relevant terms.

The AMPSIMM incorporates both ambulatory and non-ambulatory mobility, mobility in different environments, and mobility utilizing mobility aids. Therefore, it has relevance in the dysvascular population where ambulatory mobility with a prosthetic limb may or may not be achieved or when ambulatory mobility may be lost because of additional amputation or progression in multisystem disease. The AMPSIMM demonstrated that it is responsive to change when the patient improves in mobility function with or without a prosthesis. It can therefore be used to quantify mobility from time of surgery throughout the continuum of rehabilitation, and as such, offers an objective way of quantifying the impact of various rehabilitation interventions. This differs from the majority of amputee mobility measures, which focus specifically on mobility with a prosthetic limb.

Further, AMPSIMM is scored so that each numeric score is associated with a specific level of mobility in the home and/or the community, whether that level of mobility is achieved by using a wheelchair or through ambulation, and whether or not mobility aids are required. Thus, it enables clear communication of functional mobility to patient and provider. With the increased emphasis on personalized medicine and patient participation in decision making, it is important to have outcome measures that can be used in predictive models that enable clear communication of the difference in outcome associated with key clinical interventions. For example, if a prediction model informs a patient and provider that an intervention would result in a score change from 10 to 14 on a numerical scale, it would be difficult for the patient to weigh the benefits and costs of modifying the health factor. In contrast, if the intervention resulted in a change from using a wheelchair for community mobility to being ambulatory in the community, it would be conceptually easier for the risks of the intervention in relation to the effect of that intervention on outcome to be weighed by the patient. With additional research, AMPSIMM, through its structure and conceptual framework, may fulfill this important goal.

One of the fundamental obstacles to the wide-spread utilization of amputee mobility outcome measures to assess ongoing function is the clinical burden imposed by the measure and the lack of ability to interpret the data in real time. AMPSIMM does not require clinician participation and consists of a single question. Its simplicity of structure and direct linkage to daily function will allow ease of interpretation in real time.

The present data demonstrated the preliminary validity of AMPSIMM in a dysvascular amputee population. Content validity was established by ensuring that individuals with relevant clinical and methodology expertise participated in generating the content using a structured and iterative process. Concurrent and predictive validity was established by high correlations with existing measures. The construct validity of AMPSIMM was also well supported. As expected, individuals with more distal levels of amputation reported higher levels of mobility on the AMPSIMM. Similarly, higher AMPSIMM scores were associated with greater hours of prosthetic use, higher levels of mobility satisfaction, and lower levels of functional restriction. There have been a number of studies that have evaluated the psychometric properties of existing mobility outcome measures. Despite differences in the populations studied and the measures used in the validation process, the psychometric properties of the AMPSIMM appear to be similar to or better than existing measures.

### Example 3

Methods

This study used administrative, quality improvement and clinical data from two primary sources: the Veterans Affairs (VA) Surgical Quality Improvement Program (VASQIP) and the VA Corporate Data Warehouse (CDW). The study was conducted in accordance with procedures approved by the participating institution's human subjects review board.

Veterans Affairs Surgical Quality Improvement Program—The VASQIP database was used to define the inception cohort as well as several preamputation risk predictors. It includes information on 30-day surgical outcomes, and preoperative, perioperative and postoperative co-variables from 110 VA Medical Center inpatient surgical programs. VASQIP is a surgical quality improvement data set developed to monitor the quality of surgical care in the VA Health System. Data are collected on approximately 70 per cent of all major operations and about 25 per cent of all operations in the VA Health System. The process for assignment to the data collection group is random. Eligible non-cardiac procedures include those performed by a physician that require general, spinal or epidural anesthesia.

Corporate Data Warehouse—The VA CDW includes inpatient and outpatient data, as well as demographic information. Data from the CDW were used to determine whether the VASQIP surgical procedure was an incident amputation (explained below). The CDW was also used to acquire additional predictor variables (such as weight and laboratory values) that were not available through VASQIP. The CDW Vital Status File was used to identify those who died within the first year after incident amputation.

Study Sample—The target population comprised patients who survived for 1 year after undergoing their first unilateral TM, TT or TF amputation between 1 Oct. 2004 and 31 Dec. 2014 secondary to diabetes and/or PAD (based on the co-existence of ICD-9-CM codes; PAD: 440.22, 440.23, 440.24, 440.4, 442.3, 444.22; diabetes: 249.7, 250.7, 443.81, 785.4, 249.8, 250.8, 707.1, 707.11, 707.12, 707.13, 707.14, 707.15, 707.19), and were aged over 40 years. Amputation level was determined using the following codes: TM (Current Procedural Terminology (CPT) 28800, 28805; ICD-9 84.12), TT (CPT 27880, 27881, 27882, 27888, 27889; ICD-9 84.14, 84.15) or TF amputation (CPT 27590, 27591, 27598; ICD-9 84.16, 84.17, 84.18). Subjects were excluded if they had a preoperative diagnosis of coma, paraplegia, quadriplegia, disseminated cancer, tumor of the central nervous system, were ventilator-dependent, their amputation laterality could not be ascertained, or they died within 1 year of amputation. Patients were also excluded if their height, weight or BMI was considered implausible (less than 1.2 or more than 2.1 m, below 34 or above 318 kg, and less than 15 or over 52 kg/m² respectively) because these were likely to be implausible values owing to data entry errors.

These exclusions were used to create a cohort that was not at a high risk of death related to causes not typically associated with diabetes, PAD and their complications, and also to exclude those whose severe co-morbidity profile was consistent with a patient who would typically undergo a TF amputation. The goal was to create a cohort of individuals undergoing an incident lower extremity amputation for whom the most appropriate amputation level was uncertain, making the prediction model a useful tool to augment decision-making.

Definition of Incident Amputation—After defining the initial subject population using the VASQIP database, the CDW was then used to provide a look-back window between 2 days and 5 years before the amputation procedure. The presence of any diagnostic or procedure code related to amputation or its treatment resulted in the exclusion of these subjects. For guillotine procedures at the TT (CPT 27881; ICD-9 84.13) and TF (CPT 27592) levels, the presumption was that a closure procedure would be performed within 3 weeks of the guillotine procedure; therefore, research staff searched forward 3 weeks for the next procedure code to classify the incident level. If the next subsequent procedure was more than 3 weeks after the initial guillotine procedure, an error in the initial coding was presumed and the guillotine code was recorded as a definitive amputation. Some 328 patients were coded as having guillotine procedures, but did not have a subsequent closure procedure from VASQIP or CDW. For these, professional medical record annotators (professionals who review and interpret medical records for retrospective study projects) were used to identify the definitive closure level. This became the level of incident amputation.

Classification of Laterality for Incident and Subsequent Amputations—Using electronic health records from the CDW, a natural language processing (NLP) system was developed to classify the laterality of the incident amputation, sub-sequent amputation, revascularization procedures and ankle: brachial pressure index (ABPI) values. Where laterality of the amputations could not be classified using the NLP process, chart annotation was used to classify laterality. The accuracy of validation (comparison of the NLP classifications with a random sample of 100 reviewed charts) for incident amputation, subsequent amputation and revascularization was 95.0, 92.0 and 91.0 per cent respectively.

Outcome—The primary outcome of this study was ipsilateral surgical reamputation occurring within 1 year of index amputation. Reamputation was identified by an amputation procedure code in the CDW that occurred 3 weeks or more after the incident amputation and was ipsilateral to the incident procedure. Three separate categories of reamputation were identified and were all included in the study definition of reamputation: soft tissue revisions after TT (CPT 27884) and TF (CPT 27594) procedures, reamputations at the same level after TT (CPT 27886) and TF (CPT 27596) procedures, and definitive amputation at a higher level after TT (CPT 27880, 27881) and TF (CPT 27590, 27591) procedures. A search was also undertaken for ICD-9 code 84.3 representing a revision. Because TM amputations do not have codes for soft tissue revision or reamputation at the same level, all ICD-9 84.3 codes after an incident TM amputation were coded as a subsequent TM amputation, and were classified as a reamputation at the same level.

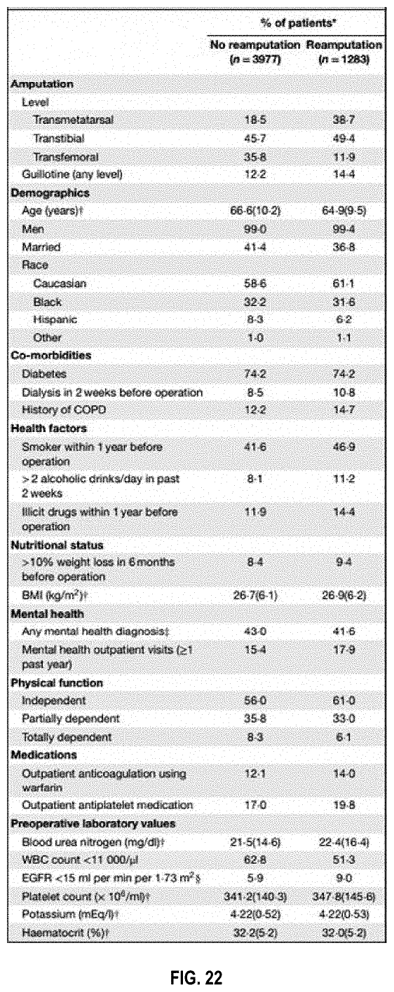

Candidate Predictor Variables—The databases described above were used to retrieve 37 potential candidate predictor variables, identified a priori through a review of the literature and expert clinical opinion; 28 variables were ultimately considered as candidates in developing a predictive model (FIG. 22) after dropping or combining nine because of a large number of missing values, difficulty in measuring that would limit future clinical utility, or because they were combined with other variables. When VASQIP laboratory values were missing, the most proximate CDW value within 3 months before the date of surgery was used; a similar process was used for missing values for other VASQIP predictors that were also recorded in the CDW. For calculation of BMI, the median CDW value for height and the VASQIP-recorded value for weight were used, as these are documented just before surgery in the hospital setting. Patients with diabetes (requiring oral agents or insulin) were identified from VASQIP. Patients not identified as having diabetes in VASQIP were classified as having diabetes if one of the ICD-9 codes listed above existed in the CDW. A well accepted, validated or standardized measure of symptomatic PAD does not exist or is very difficult to obtain from retrospective databases. Therefore, three predictors that are thought to be associated clinically with symptomatic or more severe PAD were chosen: abnormal ABPI (less than 0.9 measured in the past year), history of revascularization in the past year (open, endovascular or both), and a diagnosis of rest pain or gangrene within 30 days before surgery.

Model Development and Validation—The initial strategy was to develop a reamputation risk prediction model using patient data from the East, South and Midwest VA regions, and then to validate it externally in the West and Mountain West/Texas regions. However, geographical variability in reamputation frequency and predictor distributions led the authors to pursue the strategy of developing the model in all regions and then undertaking internal validation through bootstrapping.

There were few missing data for the 28 candidate predictor variables and so a complete-case analysis was used, as opposed to other approaches, such as multiple imputation of missing values. Candidate predictors were evaluated rigorously by univariable exploratory data analysis followed by bivariable analysis, evaluating the association between each predictor and 1-year reamputation. Twelve potential interactions based on clinical experience were considered. Seven with TM amputation (married, smoking, alcohol abuse, illicit drug use, diabetes, kidney failure (based on estimated glomerular filtration rate (eGFR) values), and rest pain/gangrene), one with TT amputation (guillotine amputation), one with TF amputation (smoking) and three with diabetes (abnormal ABPI, rest pain/gangrene and history of revascularization). Associations with continuous predictors were assumed to be linear (on the logit scale).

A logistic regression model with all 28 candidate predictors and 12 interaction terms was fitted to pro-vide a reference for comparison with more parsimonious models. Variable selection for more parsimonious prediction models using both backward stepwise and stepdown logistic regression methods was considered. For the backwards stepwise variable selection approach, a P value cut-off of 0.157 was chosen, which approximates the best subset of predictors using the Akaike information criterion. For the stepdown variable selection approach, models that explained 99 and 95 per cent of the variability in the risk predictions from the full model were considered. Calibration was evaluated by means of the Hosmer—Lemeshow (H-L) goodness-of-fit test. A plot of observed fraction of 1-year reamputation versus the mean of model-estimated risks of 1-year reamputation for each decile of predicted risk was also assessed visually. Discrimination was assessed quantitatively by calculating the area under the receiver operating characteristic curve (AUC), the discrimination slope (difference in mean predicted 1-year reamputation probabilities for those who did and those who did not undergo reamputation within the first year), and the difference in mean estimated risk in the highest and lowest deciles of predicted risk.

The final model was validated internally with bootstrap sampling to obtain estimates of the optimism (discrepancy due to overfitting) of the estimated AUC and the difference in predicted probabilities for those who did or did not undergo reamputation (discrimination slope). Bootstrap samples were drawn with replacement and with the same size as the original sample. Model selection was carried out for each bootstrap sample and model performance assessment compared with that of the original sample. This was repeated 500 times to obtain stable estimates of the mean optimism of the AUC and discrimination slope for each model.

Results

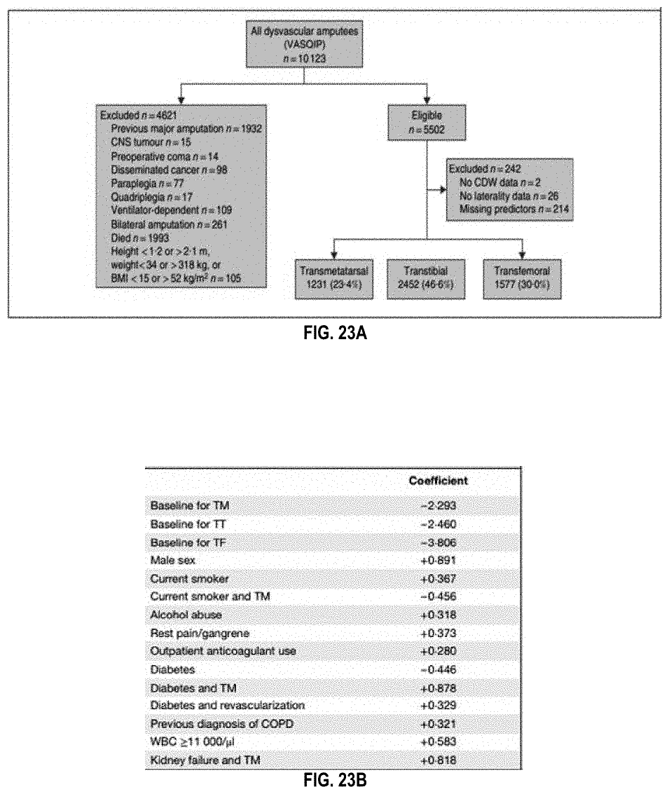

The complete-case cohort included 5260 individuals who underwent lower extremity amputation owing to the complications of PAD and/or diabetes. There were 1231 TM (23.4 per cent), 2452 TT (46.6 per cent) and 1577 TF (30.0 per cent) amputees (FIG. 23A). This represented 95.6 per cent of those eligible. The distributions of the 28 candidate predictors evaluated in the prediction model are summarized by reamputation status in FIG. 22.

Outcomes—A total of 1283 ipsilateral reamputations (24.4 per cent) occurred within the first year after incident amputation. The risk of ipsilateral reamputation was 40.3 per cent among TM amputees (496 of 1231), 25.9 per cent in TT amputees (634 of 2452) and 9.7 per cent in TF amputees (153 of 1577).

Risk Prediction Model Development—The backwards stepwise variable selection resulted in a model with 16 variables. The 99 per cent stepdown variable selection resulted in a model with 19 variables and the 95 per cent stepdown variable selection resulted in a model with 11 variables. These 11 variables were a subset of the 16 variables from the backwards stepwise model. The three predictor models were compared with the full model as the criterion standard. All models performed similarly to the full model with respect to qualitative (graphical) and quantitative (AUC and discrimination slope) assessments. Given this, the 11-variable model was chosen (FIG. 23B), as it was more parsimonious, resulting in a lower clinical burden for the provider to obtain the information. This 11-predictor model contained nine main effects and four interactions. FIG. 23B shows the components of the modelled reamputation risk score. Both TM and TT amputees were at higher risk of 1-year reamputation than TF amputees. The modelled reamputation risk in TT and TM amputees was similar, except for TM amputees who had diabetes or were in kidney failure (based on eGFR values), who had a higher risk. Other predictive factors associated with increased risk of reamputation were current smoking in TT and TF amputees, alcohol abuse, presence of rest pain/gangrene, use of outpatient anticoagulants, previous diagnosis of chronic obstructive pulmonary disease, and white blood cell counts of 11000 /μl or higher. Having both diabetes and a history of an ipsilateral revascularization in the previous year increased the risk for TM amputees.

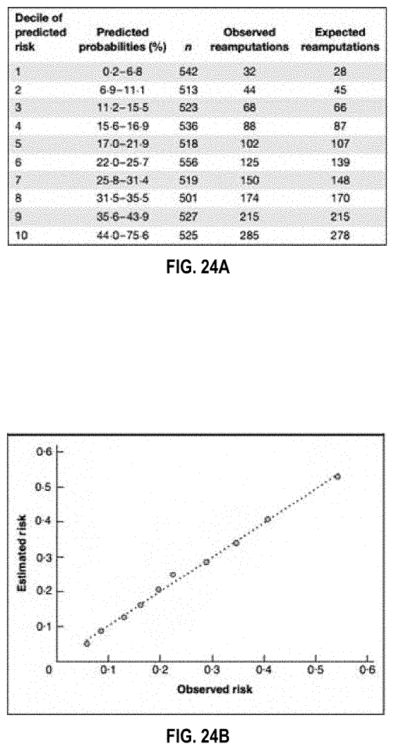

The mean 1-year reamputation risk in the cohort of 5260 patients predicted using the 11-variable risk prediction model was 24.4 (range 2.0 to 75.6) per cent. The estimated AUC was 0.72 and the H-L goodness-of-fit test for this model indicated good calibration (P=0.918) (FIG. 24A). The discrimination slope was 11.2 per cent. The difference in mean predicted risk of reamputation in the highest versus lowest deciles of predicted risk was 47.7 per cent (FIG. 24B).

Internal Validation of Risk Prediction Model—Bootstrap estimates of the optimism for both the AUC and discrimination slope were 0.01, indicating that there was negligible optimism in these estimated accuracy characteristics when using the same data to develop and validate the model.

Discussion

A novel reamputation risk prediction model (AMPREDICT Reamputation) was developed and validated internally in this study. It can be used to quantify the individual risk of reamputation within 1 year of the incident amputation procedure in patients who require TM, TT or TF amputation because of complications of diabetes and/or PAD, and who survive the first year after incident amputation. The model was developed using a large data set of 5260 individuals undergoing amputation, of whom 1283 had an ipsilateral reamputation in the subsequent year. The model demonstrated good prediction characteristics which confirmed its potential in communicating individual patient risk. The predicted reamputation risks vary substantially based on an individual's risk factors. Therefore, the amputation-level choice surgeons and their patients make may differ considerably depending on how the magnitude of risk and the downstream consequences of this risk are viewed.

Other studies have indicated that the risk of reamputation is dependent on amputation level, increasing the more distal the amputation. TM amputation has been associated with a 29-35 per cent reamputation risk, whereas TT and TF amputations have been associated with risk of 12-25 per cent and 8 per cent respectively. In addition, multiple health factors have been shown to affect reamputation risk, including male sex, smoking, alcohol use, rest pain/gangrene, anticoagulant and aspirin use, increased international normalized ratio, increased white blood cell count/sepsis and history of revascularization. These summaries inform population risk, but do not inform individual patient risk, which is essential for incorporation into treatment planning decisions.

Informing patients and surgeons about individual reamputation risk is critical because of its effect on key health and quality-of-life outcomes. Reamputation surgery after TM amputation imposes an additional mortality risk, which has been estimated at 5 per cent. In a mixed population of TM, TT and TF amputees, who required hospital readmission primarily for wound-related complications, there was a twofold increase in risk of death. In addition to increased mortality risk, failure of primary healing is complicated by the need for additional wound care, hospital admissions, and often multiple operations which result in a substantial burden to patients and families. There is also concern that reamputation may lead to a reduction in mobility and quality of life that exceeds that of an initial higher-level amputation. The extremely poor 1- and 5-year survival rates after dysvascular amputation also create an imperative to better inform patients about the outcome risks associated with amputation at each level, so that their personal priorities regarding how they wish to spend their remaining life are incorporated into the surgical decision.

The AMPREDICT Reamputation prediction model is a novel tool that could be used in conjunction with a previously published mobility prediction model and mortality prediction model to provide an individualized multidimensional view of key outcomes associated with each amputation level. These tools can be used to balance the risks and benefits of other key outcomes, including body image, social stigma and self-esteem, in making the amputation-level choice. Patients want to participate in the amputation-level decision and want to be better informed. The proposed reamputation risk prediction model, in conjunction with the mobility and mortality risk prediction models, enables an assessment of individual-patient and amputation-level risk that can be used to better inform patient and surgeon, so they can develop an individualized plan of care that incorporates patient priorities in arriving at an amputation-level decision. When such assessments are used in a shared decision-making environment, patients will be better informed, which should result in more value-concordant healthcare decisions. Patient involvement and empowerment may result in more positive health and psychological outcomes.

This model has a number of strengths as well as potential limitations. The validity of the model is supported by the agreement between the predictors identified in this model and factors that have been associated with reamputation risk in previous studies. From a methodological perspective, the study has strengths in its approach to defining reamputation; previous studies used ICD-9 codes that do not define laterality or CPT laterality modifier codes, which raises uncertainty regarding whether the second procedure was ipsilateral or contralateral to the initial surgery. To overcome this limitation, NLP and chart annotation were employed to ensure that subsequent amputations were ipsilateral to the initial amputation. Furthermore, guillotine amputations are also commonly employed as an emergency procedure to control infection, with a planned subsequent definitive amputation. The amputation after the guillotine procedure was defined as the definitive amputation level, and only the next subsequent ipsilateral amputation was counted as a reamputation. One primary limitation is the generalizability of the findings. Some caution is advisable when applying the model to women, Hispanics and patients who fall into the ‘other’ race category, such as Asians, Pacific Islanders and Native Americans, owing to the limited number of subjects in these categories. Patients who experienced a previous major amputation or had severe co-morbidities that typically lead to TF amputation were excluded, as this study was interested in a cohort of patients in whom the amputation decision level would be less certain. Only patients who survived the first year after amputation were included; as mortality is a competing risk with reamputation, this study sought to predict the risk of reamputation among those who survive. The model was also developed using a specific population comprising US veterans exclusively, and external validation in other populations is needed. However, prediction models developed for other purposes in VA populations have shown strong external validation characteristics with non-veteran populations. Potential predictors with a large number of missing values such as albumin levels and haemoglobin (Hb) A1c levels were dropped from consideration. Interestingly, neither albumin nor HbA1c levels were associated with reamputation in bivariable assessments among subjects for whom these variables were recorded (data not shown). Despite reasonable discrimination and good calibration, the discrimination slope of 11.2 per cent is modest, probably because 90 per cent of the predicted reamputation probabilities for the cohort were below 44 per cent (FIG. 24A). Some of this may be explained by unmeasured characteristics in these patients that the study was unable to account for and inclusion of which might have further improved the performance of the model. Some examples include patient frailty and social support structures; however, marital status was included as a surrogate for social support, and independence in self-care as a potential surrogate for frailty. Neither of these surrogate measures affected reamputation risk. The challenge in evaluating any potential effect of these variables on model enhancement is that there would be a need for widespread agreement on which specific frailty and social support measures to use, and to have them implemented routinely as part of patient care practices. Because this prediction model is designed to inform probable risk of reamputation at the time of surgical decision-making, it does not incorporate operative or postoperative factors which may modify the risk of healing. Finally, as with all prediction models, this model requires periodic recalibration to ensure adequate future performance.

With completion of the development of validated pre-diction models for mobility, mortality and reamputation, future work is needed to determine how best to communicate these risks to patients using patient decision aids, and to surgeons using clinical decision support tools. Implementation studies will be critical to help ensure that these models can be incorporated into clinical care pathways and are clinically useful.

### Example 4

Methods

Veterans Affairs Surgical Quality Improvement—The Veterans Affairs Surgical Quality Improvement Program (VASQIP) database was used to define the inception cohort as well as several preamputation risk predictors. It includes information on 30-day surgical outcomes, and preoperative, perioperative and postoperative co-variables from 110 Veterans Affairs Medical Center inpatient surgical programs. VASQIP is a surgical quality improvement data set developed to monitor the quality of surgical care in the Veterans Affairs (VA) Health Care System. Data were collected on approximately 70 per cent of all major operations and about 25 per cent of all operations in the VA Health Care System. The calendar is subdivided into consecutive 8-day cycles, each starting on a different day of the week. In each 8-day cycle, the first 36 consecutive eligible surgical patients in that cycle are entered into VASQIP. Eligible non-cardiac procedures include those performed by a physician that require general, spinal or epidural anesthesia.

Corporate Data Warehouse—The VA Corporate Data Warehouse (CDW) includes inpatient and outpatient data as well as demographic information. Data from the CDW were used to assess whether individuals had previously undergone an amputation procedure (and were thus ineligible for the present study) and to acquire additional predictor variables (such as weight and laboratory values) that were not available through VASQIP. The CDW's Vital Status File was used to determine date of death of those who died.

Study Sample—The target population was patients undergoing their first unilateral TM, TT or TF amputation assumed to be secondary to diabetes and/or PAD (based on ICD-9-CM codes; diabetes: 249.7, 250.7, 785.4, 443.81, 785.4, 249.8, 250.8, 707.1, 707.11, 707.12, 707.13, 707.14, 707.15, 707.19; PAD: 440.22, 440.23, 440.24, 440.4, 442.3, 444.22). VASQIP subjects were included in the study cohort if they were aged 40 years or older (to ensure that the study population was restricted to those undergoing amputations related to diabetes and/or PAD15) and had undergone an incident major unilateral lower extremity amputation, defined as TM (Current Procedures Terminology (CPT): 28800, 28805; ICD-9: 84.12), TT (CPT: 27880, 27881, 27882, 27888, 27889; ICD-9: 84.14, 84.15) or TF (CPT: 27590, 27591, 27598; ICD-9: 84.16, 84.17, 84.18) amputation between 1 Oct. 2004 and 31 Dec. 2014. Those who had a record of a previous major lower extremity amputation in the CDW occurring between 2 days and 5 years before the VASQIP operation were excluded. Subjects were also excluded if they had a preoperative diagnosis of coma, paraplegia, quadriplegia, disseminated cancer, a tumor of the central nervous system, or were ventilator-dependent, because these conditions result in atypical risks of death which may more commonly lead to a higher level of amputation. It was presumed that patients undergoing a guillotine procedure at the TT (CPT: 27881; ICD-9: 84.13) and TF (CPT: 27592) levels would have a closure procedure within 3 weeks of the guillotine procedure; therefore, research staff searched forward 3 weeks for the next procedure code to classify the incident amputation level. If the next subsequent procedure occurred more than 3 weeks after the initial procedure, it was presumed that the initial guillotine code was an error, and the initial guillotine procedure was accepted as a definitive level of amputation and any subsequent procedure as a reamputation. For those coded as having guillotine procedures, without a subsequent closure procedure from VASQIP or CDW, VA Informatics and Computing Infrastructure chart annotation services were used to identify the definitive closure level and laterality.

Outcome—The primary outcome of this study was death within 1 year of index amputation.

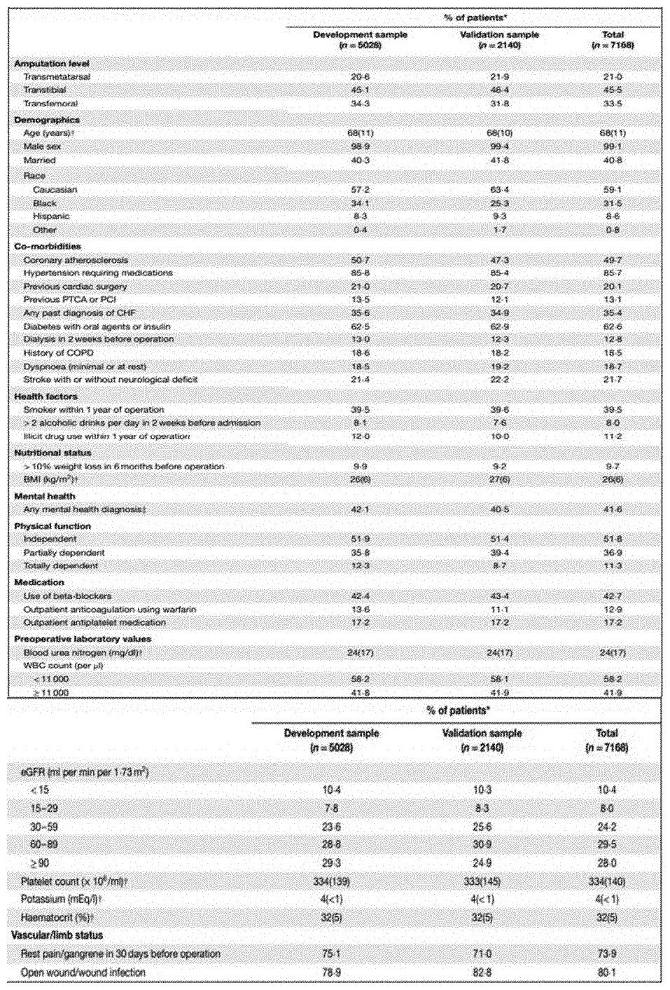

Candidate Predictor Variables—The databases were used to retrieve 50 potential predictor variables, identified a priori from evidence in the literature and expert clinical opinion, based on an informal process that included four epidemiologists (1 an expert in VASQIP data, another in CDW data and 2 with amputee research experience), a physical medicine physician with national recognition in amputation care and research, a vascular surgeon, an internal medicine physician with clinical and research expertise in diabetic foot ulceration and amputation risk, and a rehabilitation psychologist. Thirty-three variables were ultimately considered as candidates in developing a predictive model (FIG. 25), after dropping or combining 17 variables because of low frequency of occurrence, difficulty in clinical measurement, or because they were combined with other variables. When VASQIP laboratory values were missing, the nearest CDW value within 3 months before the date of surgery was used; missing values for other VASQIP predictors that were also recorded in the CDW were dealt with similarly. To calculate BMI, the median CDW value for height and the VASQIP recorded value for weight were used, as these are documented just before surgery in the hospital setting. Patients were excluded if their height, weight or BMI was considered implausible (less than 1.2 or more than 2.1 m; below 34 or above 318 pound; and less than 15 or over 52 kg/m²) because these represent patients who may be severely malnourished or obese and may be more likely to be a candidate for a TF amputation, and/or there has been a data entry error.

Model Development and Validation Samples—Geographical validation was used to validate the prediction model externally. The prediction model was developed using three of the five regions within VA (East, South and Midwest), and validated in the Mountain plus Texas and West regions. The selection of regions was based both on geography (dividing the USA nearly in half) and on sample size, allowing a larger sample size in the development (training) cohort.

Statistical Analysis—The 33 candidate predictors were evaluated rigorously through exploratory data analysis followed by bivariable analysis, analyzing the association between each predictor and 1-year mortality. There were few missing data for these candidate variables and so a complete-case analysis was used as opposed to other approaches, such as multiple imputation of missing values. Interactions were not considered among the candidate predictors because biological plausibility could not be justified, nor, after a thorough review of the literature, could any evidence for such interactions be identified. For all continuous measures, non-linearity of the association with (logit) risk of 1-year mortality was explored graphically using non-parametric smoothing and by comparison of linear with fractional polynomial models.

A full logistic regression model with all 33 candidate predictors was fitted to provide a reference for comparison with more parsimonious models. Variable selection using both backwards stepwise and stepdown logistic regression methods was considered. For the backwards stepwise variable selection approach, a P value cut-off of 0.157 was chosen, which has been shown to approximate the best subset of predictors using the Akaike information criterion. For the stepdown variable selection approach, models that explained 99 and 95 per cent of the variability in the risk predictions from the full model were considered. Calibration and discrimination of the fitted models were assessed separately in the development (training) and validation samples. Calibration was assessed by means of the Hosmer-Lemeshow (H-L) goodness-of-fit test and Cox calibration regression. A plot of observed fraction of 1-year deaths versus the average of predicted risks of 1-year death for each decile of predicted risk was assessed visually. Discrimination was assessed quantitatively by calculating the area under the receiver operating characteristic (ROC) curve (AUC), the discrimination slope (the difference in mean predicted 1-year mortality risk for those who did and those who did not die within the first year) and the difference in mean estimated mortality risk in the highest and lowest deciles of predicted risk.

Results

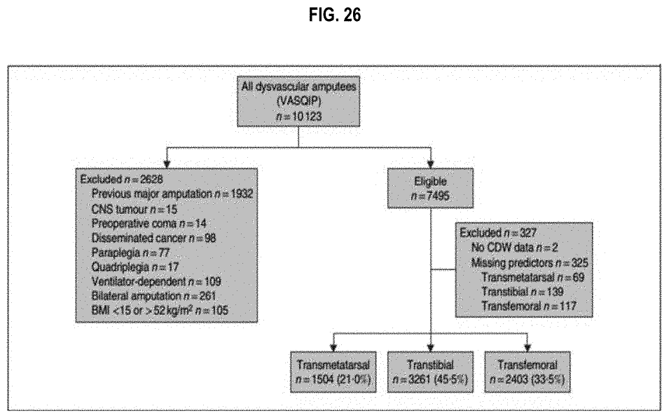

Of 7495 eligible people, two with no CDW data were excluded, leaving 7493 (East, 1896; South, 1849; Midwest, 1459; Mountain plus Texas, 1139; West, 1150). Some 325 individuals were missing at least one observation and therefore excluded. The data set analyzed included 7168 people with non-traumatic amputation owing to diabetes and/or PAD: 1504 TM amputees (21.0 per cent), 3261 TT amputees (45.5 per cent) and 2403 TF amputees (33.5 per cent) (FIG. 25, FIG. 26). This represented 95.6 per cent of those eligible.

Outcome—Some 1901 patients (26.5 per cent) died within the first year after incident amputation. The mortality risk increased by amputation level (TM, 17.7 per cent; TT, 24.7 per cent; TF, 34.5 per cent).

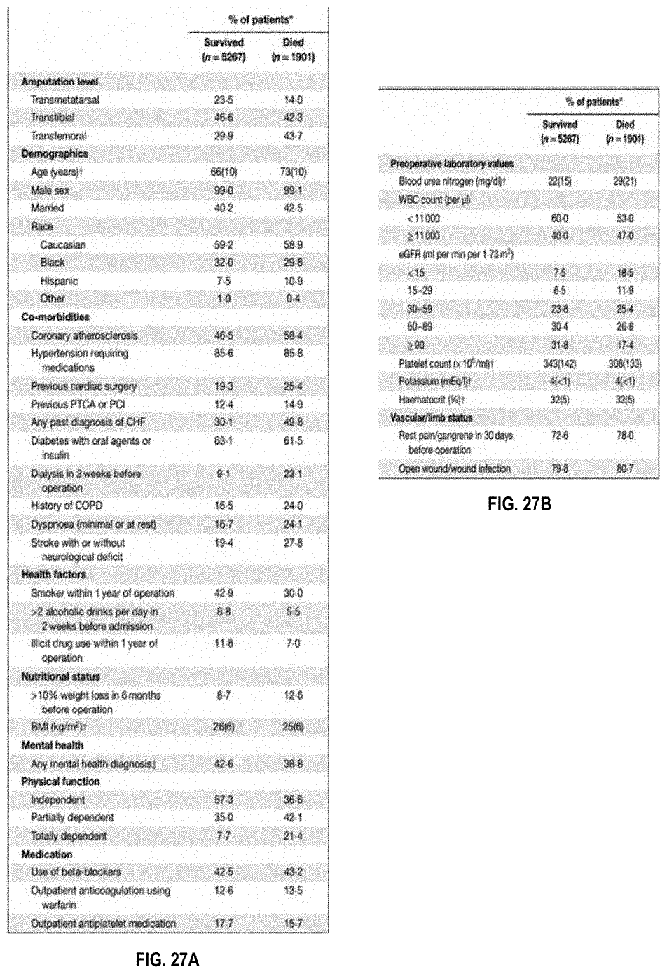

Risk Prediction Model Development—The development sample consisted of 5028 subjects from the East, South and Midwest VA regions. Platelet count and serum potassium concentration were modelled in a non-linear manner using fractional polynomials. Estimated glomerular filtration rate and white blood cell counts were divided into clinically relevant categories. Other continuous predictors were modelled linearly and categorical factors as binary variables (yes/no) unless specified otherwise (FIG. 27).

The backwards stepwise and 99 per cent stepdown variable selection resulted in the same model with 17 predictor variables, whereas the 95 per cent stepdown variable selection resulted in a model with ten predictor variables (a subset of the 17 predictors). The 17- and ten-predictor models were compared with the full model as the criterion standard. Both models performed similarly to the full model with respect to qualitative (graphical) and quantitative (AUC and discrimination slope) assessments. The ten-predictor model was chosen, as it was more parsimonious and would result in a lower clinical burden for the provider to obtain the information. The mean of the 1-year mortality risks predicted for each individual in the development sample using the ten-predictor risk prediction model was 26.5 (range 0.7-94.9) per cent. The estimated AUC was 0.77 and the H-L goodness-of-fit test for this model indicated good calibration (P=0.523). The discrimination slope was 18.9 per cent.

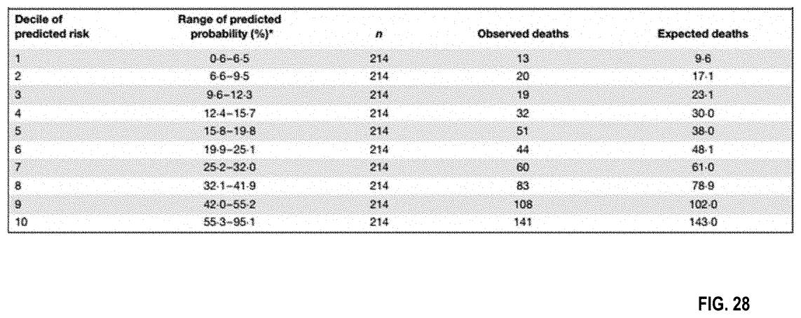

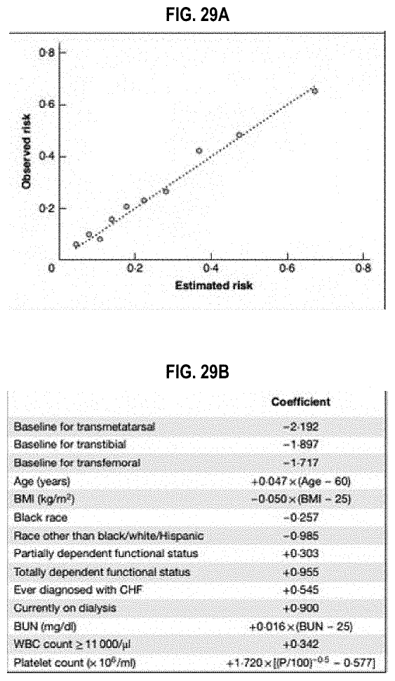

Risk Prediction Model Validation—The external validation sample consisted of 2140 subjects from the Mountain plus Texas and West regions (FIG. 25). The overall risk of death in the validation sample was 26.7 per cent. The mean predicted risk of 1-year mortality using the ten-predictor risk prediction model was 25.7 (range 0.6-95.1) per cent. (FIG. 28 and FIG. 29A summarize observed versus predicted risks of 1-year mortality by deciles of predicted risk. The fit of the model to the validation sample was good, and this was further supported by the H-L goodness-of-fit test result (P=0.283). The estimated AUC for the validation sample was 0.76 and Cox calibration regression yielded an estimated slope of 0.96 (95 per cent c.i. 0.85 to 1.06) and intercept of 0.02 (−0.12 to 0.17), again indicating good calibration (perfect calibration is represented by a slope of 1.00 and intercept of 0). The discrimination slope was 18.2 per cent. The difference in mean estimated risk of death in the highest versus lowest deciles of predicted risk was 62.3 per cent.

Combined Risk Prediction—Given the strong external validation characteristics, the development and validation samples were combined (FIG. 29B). Amputation level was associated with 1-year mortality, increasing amputation levels being associated with a greater risk of death. Other predictive factors associated with increased risk of death were older age, partially and totally dependent functional status, ever being diagnosed with congestive heart failure, being currently on dialysis, increasing blood urea nitrogen levels, and white blood cell counts of at least 11 000 /μl. Individuals with greater BMI, and those who were black or in the ‘other’ category for race were at a decreased risk of death. There was a non-linear relationship between platelet levels and risk of death, with a rapid decrease in risk with increased platelet levels, until approximately 400×10⁶/ml, and then stabilizing thereafter. The modelled mortality risk equation in FIG. 29B can be employed easily for individual mortality risk calculations.

Discussion—A 1-year mortality risk prediction model, AMPREDICT-Mortality, was developed in a population of patients who experienced an incident lower extremity amputation at the TM, TT or TF levels secondary to diabetes and/or PAD. The model has good calibration characteristics and has been validated externally using VA regional data. The developed model can be used to inform surgeons and their patients about individual-patient mortality risk.

Patients facing amputation benefit from, and want more information about, the anticipated outcome of their surgery, and want to participate in treatment decisions. Mortality risk is a key outcome that should be considered at the time of amputation-level decision-making. Individuals who have undergone lower extremity amputation owing to diabetes and/or PAD have 1- and 5-year mortality risks that exceed those of most cancers; they can be as high as 44 and 77 per cent respectively. The limited lifespan of these patients creates an additional imperative to ensure that treatment decisions allow them best to achieve their personal outcome priorities and optimal quality of life, for their remaining life. A knowledge of individual mortality risk is therefore important in tailoring medical and surgical decisions. Knowing which patients have a high or low risk of death in the first year after amputation will help surgeons better assist patients in making end-of-life decisions and also making more informed treatment decisions that incorporate their values and goals.

Mortality risk is certainly not the sole factor to consider in the treatment decision. It is, however, an outcome that acts as a foundation for patients and surgeons to balance the risks of other key outcomes such as mobility, further ipsilateral amputation, quality of life, and the body image consequences of each amputation level. For example, a patient with an 80 per cent risk of 1-year mortality may balance the risk of additional surgery owing to failure of healing of a TM amputation differently than if they had a 20 per cent risk of death.

There are a number of limitations of the study. With respect to the generalizability of the findings, because the model was developed and validated in a US population that comprised predominantly older Caucasian men, some caution is advisable when applying the model outside of the USA, and to women, Hispanic people and patients who fall into the ‘other’ race category such as Asians, Pacific Islanders and Native Americans. Patients aged less than 40 years were excluded to avoid including patients who may have been misclassified. The VASQIP data set does not include amputations that were performed with locoregional anesthesia, and patients with such amputations were not therefore included in the present study cohort. However, the proportion of TM amputations in this cohort closely resembles that reported by the VA over the same 15-year interval; therefore, it is unlikely that TM amputations were under-represented. It is also unlikely that differences in patient characteristics that may be associated with this type of anesthetic will confound the results. Therefore, the present mortality risk prediction model should not be used for patients undergoing revisional amputation surgery, contralateral amputations and for patients who have a high risk of death from causes other than those typically seen in diabetes and PAD, such as people with metastatic cancer. Further study is needed to determine the applicability of the mortality risk prediction model in other populations. Potential predictors that were rare (present in less than 5 per cent of the study cohort) were not included in the modelling. This included amputations such as knee and ankle disarticulastions and Syme's procedures, as well as rare co-morbidities such as acute renal injury and recent myocardial infarction. Predictors with a large number of missing values such as albumin levels, ejection fraction (EF) and HbA1c levels were also dropped from consideration. Interestingly, neither albumin levels, EF nor HbA1c levels were associated with mortality in bivariable assessments of subjects for whom measurements were available. Although the predictive characteristics of this model are good, there may be some unmeasured characteristics that the study was unable to account for, and which might have further improved the performance of the model.

Although previous studies have estimated 1-year mortality, the modelling approach used here differs from that of other prediction models. The present AMPREDICT-Mortality model permits estimation of mortality risk for each amputation level in a single parsimonious model, in a relatively homogeneous population.

Furthermore, this study limited the target population to those with no previous amputations, and excluded co-morbidities strongly associated with mortality and a clinical indication for a TF amputation. Previous prediction models, with the exception of those reported by Nelson and colleagues, who developed separate models for TT and TF amputations, predicted mortality risk of all patients undergoing a ‘major amputation’, and the effects of amputation level on risk were not modelled. These models also included patients who may have undergone multiple ipsilateral or contralateral amputations previously. The mortality risks of patients included in the sample used to develop those models may therefore differ significantly from those of patients undergoing their first amputation. Another important difference in the present prediction model is that the TM amputation level was included, acknowledging the increasing rate at which these procedures are being performed to salvage most of the foot. Furthermore, the time interval of analysis of the present model was extended from the perioperative period (30 days after surgery) to 1 year. A 1-year time frame was chosen because of its greater relevance to survival rather than exclusively focusing on operative mortality. Finally, to optimize the potential clinical value of the model, this study aimed for parsimony and selection of readily available model predictors, while maintaining good discrimination characteristics.

While specific configurations have been described, it is not intended that the scope be limited to the particular configurations set forth, as the configurations herein are intended in all respects to be possible configurations rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
receiving patient data comprising one or more patient predictor variables;
determining, based on the patient data, one or more predictor models comprising one or more population predictor variables, wherein the one or more population predictor variables are associated with the one or more patient predictor variables;
determining, based on the one or more predictor models and the one or more patient predictor variables, one or more patient risk scores, wherein the one or more patient risk scores are associated with a predictor model of the one or more predictor models; and
outputting the one or more patient risk scores.

2. The method of claim 1, wherein the patient data comprises an electronic health record.

3. The method of claim 1, wherein the one or more population predictor variables comprise data associated with a plurality of electronic health records.

4. The method of claim 1, wherein the one or more predictor models comprise at least one of a reamputation risk model, a mobility risk model, or a mortality risk model.

5. The method of claim 1, wherein determining the one or more predictor models comprises:
selecting at least one of a reamputation risk model, a mobility risk model, or a mortality risk model from a database; and
inputting the patient data.

6. The method of claim 1, wherein determining the one or more patient risk scores comprises determining, based on the one or more predictor models, one or more patient predictor variable coefficients.

7. The method of claim 1, wherein outputting the one or more patient risk scores comprises:
sending, to a display device, the one or more patient risk scores; and
displaying, via the display device, the one or more patient risk scores.

8. A system comprising:
a computing device configured to:
receive patient data comprising one or more patient predictor variables;
determine, based on the patient data, one or more predictor models comprising one or more population predictor variables, wherein the one or more population predictor variables are associated with the one or more patient predictor variables;
determine, based on the one or more predictor models and the one or more patient predictor variables, one or more patient risk scores, wherein the one or more patient risk scores are associated with a predictor model of the one or more predictor models; and
a display device configured to:
output the one or more patient risk scores.

9. The system of claim 8, wherein the patient data comprises an electronic health record.

10. The system of claim 8, wherein the one or more population predictor variables comprise data associated with a plurality of electronic health records.

11. The system of claim 9, wherein the one or more predictor models comprise at least one of a reamputation risk model, a mobility risk model, or a mortality risk model.

12. The system of claim 9, wherein the computing device is further configured to:
selecting at least one of a reamputation risk model, a mobility risk model, or a mortality risk model from a database; and
inputting the patient data.

13. The system of claim 9, wherein the computing device configured to determine the one or more patient risk scores is further configured to determine, based on the one or more predictor models, one or more patient predictor variable coefficients.

14. The system of claim 9, wherein the display device configured to out output the one or more patient risk scores is further configured to:
display the one or more patient risk scores.

15. An apparatus, comprising:
one or more processors; and
memory storing processor executable instructions that, when executed by the one or more processors, cause the apparatus to:
receive patient data comprising one or more patient predictor variables;
determine, based on the patient data, one or more predictor models comprising one or more population predictor variables, wherein the one or more population predictor variables are associated with the one or more patient predictor variables;
determine, based on the one or more predictor models and the one or more patient predictor variables, one or more patient risk scores, wherein the one or more patient risk scores are associated with a predictor model of the one or more predictor models; and
output the one or more patient risk scores.

16. The apparatus of claim 15, wherein the patient data comprises an electronic health record.

17. The apparatus of claim 15, wherein the one or more predictor models comprise at least one of a reamputation risk model, a mobility risk model, or a mortality risk model.

18. The apparatus of claim 15, where in the processor executable instructions that, when executed by the one or more processors, cause the apparatus to determine the one or more predictor models, further cause the apparatus to:
selecting at least one of a reamputation risk model, a mobility risk model, or a mortality risk model from a database; and
inputting the patient data.

19. The apparatus of claim 15, wherein the processor executable instructions that, when executed by the one or more processors, cause the apparatus to determine the one or more patient risk scores further cause the apparatus to determine, based on the one or more predictor models, one or more patient predictor variable coefficients.

20. The apparatus of claim 15, wherein the processor executable instructions that, when executed by the one or more processors, cause the apparatus to output the one or more patient risk scores, further cause the apparatus to:
send, to a display device, the one or more patient risk scores; and
display, via the display device, the one or more patient risk scores.
